(19) United States
(12) Patent Application Publication
(10) Pub. No.: US 2026/0315104 A1
(43) Pub. Date: Oct. 15, 2026

(54) DEVICES AND METHODS FOR ANCHORING CONFIRMATION

(71) Applicant: Edwards Lifesciences Corporation, Irvine, CA (US)

(72) Inventors: Peleg Harel, Haifa (IL); Dmitry Pinhasov, Yokneam Illit (IL); Eitan Atias, Tel Aviv (IL); Ofir Witzman, Harish (IL); Halit Yaakobovich, Netanya (IL)

(73) Assignee: Edwards Lifesciences Corporation, Irvine, CA (US)

(21) Appl. No.: 19/695,159

(22) Filed: Jun. 2, 2026

Related U.S. Application Data
(63) Continuation of application No. PCT/US2024/058862, filed on Dec. 6, 2024.
(60) Provisional application No. 63/610,909, filed on Dec. 15, 2023.

(51) Int. Cl. A61B 17/34; A61B 90/00

(52) U.S. Cl. CPC A61B 17/3417; A61B 17/3468; A61B 90/39; A61B 2017/3488; A61B 2090/3966

(57) ABSTRACT

The present disclosure relates to tissue perforation devices and methods for use thereof. In an example, a tissue perforation apparatus comprises an anchor device having a helical anchor and a plunger disposed around the helical anchor. The plunger and the helical anchor are axially movable relative to each other. The apparatus further includes a radiopaque anchor marker configured to move along with the helical anchor, and a radiopaque plunger marker configured to move along with the plunger, wherein an axial distance between the radiopaque anchor marker and the radiopaque plunger marker can be indicative of successful anchoring of the helical anchor to a target tissue.

### CROSS-REFERENCE TO RELATED APPLICATIONS

This application is a continuation of International Application No. PCT/US 2024/058862, filed Dec. 6, 2024, which claims the benefit of U.S. Provisional Application No. 63/610,909, filed Dec. 15, 2023, which is incorporated by reference herein.

### FIELD

The present disclosure relates to devices and systems configured to form an opening in a target tissue, and to devices and method for confirming anchoring of a helical anchor to the target tissue.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (for example, stents) and artificial valves, as well as a number of known methods of implanting these devices and valves in humans. Percutaneous and minimally-invasive surgical approaches, such as transcatheter aortic valve replacement (TAVR), are used in various procedures to deliver prosthetic medical devices to locations inside the body that are not readily accessible by surgery or where access without surgery is desirable.

Transcatheter aortic valve replacement (TAVR) is one example of a minimally-invasive surgical procedure used to replace a native aortic valve. In one specific example of the procedure, an expandable prosthetic heart valve is mounted in a crimped state on the distal end of a delivery apparatus and advanced through the patient's vasculature (for example, through a femoral artery and the aorta) to the heart. The prosthetic heart valve is positioned within the native valve and expanded to its functional size.

A variant of TAVR is valve-in-valve (ViV) TAVR, where a new prosthetic heart valve replaces a previously implanted prosthetic valve. In one specific example of the procedure, a new expandable prosthetic heart valve (“guest valve”) is delivered to the heart in a crimped state, as described above for the “native” TAVR. The guest valve is positioned within the previously implanted prosthetic valve (“host valve”) and then expanded to its functional size. The host valve in a ViV TAVR procedure can be a surgically implanted prosthetic valve or a transcatheter prosthetic valve. The term “host valve” is also used herein to refer to the native aortic valve in a native TAVR procedure.

### SUMMARY

Needles can be utilized for piercing existing leaflets to form an opening that modifies the existing valvular structure, after which a guest prosthetic valve can be implanted in the modified valvular structure, mitigating the risk of coronary ostial obstruction. A helical anchor can be anchored to the leaflet prior to piercing thereof by the needle, to stabilize the leaflet during advancement thereof through the tissue. In some cases, an attempt to anchor the helical anchor to the leaflet may fail, leaving the entirety of the helical anchor above the leaflet, such that advancement of the needle thereafter may push the leaflet against the tip of the needle and/or cause a sliding movement therebetween, such that even if the needle eventually penetrates through the tissue, the site of penetration can be offset from the desired position along the target leaflet. Thus, there is a need to provide devices and method by which successfully anchoring of the helical anchor to the leaflet can be confirmed prior to needle advancement.

According to some aspects of the disclosure, there is provided a tissue perforation apparatus comprising: an anchor device comprising: a helical anchor, a plunger, a radiopaque anchor marker, and a radiopaque plunger marker. The helical anchor defines an anchor channel and terminates with an anchor tip. The plunger extends between a plunger distal end portion and a plunger proximal end portion.

In some examples, the radiopaque anchor marker is configured to move along with the helical anchor.

In some examples, the plunger is disposed around at least a portion of the helical anchor, wherein the plunger and the helical anchor are axially movable with respect to each other.

In some examples, the radiopaque anchor marker is configured to move along with the helical anchor.

In some examples, the radiopaque plunger marker is configured to move along with the plunger.

In some examples, the helical anchor is configured to move distally relative to the plunger when the helical anchor is anchored to a target tissue.

In some examples, an axial distance between the radiopaque anchor marker and the radiopaque plunger marker is configured to change when the helical anchor is anchored to a target tissue.

In some examples, the apparatus further comprises a biasing member configured to bias the plunger to a distally-biased position relative to the helical anchor.

In some examples, the anchor device further comprises a connector disposed between the helical anchor and the anchor shaft.

In some examples, the connector comprises a connector distal end portion affixed to the helical anchor, a connector proximal end portion affixed to the anchor shaft, and a connector intermediate portion extending between the connector distal end portion and the connector proximal end portion.

In some examples, a distal end of the connector defines a connector distal outer step at a transition between the connector distal end portion and the helical anchor.

In some examples, the plunger defines a plunger distal step distal to the connector distal outer step.

In some examples, the biasing member is disposed between the plunger distal step and the connector distal outer step.

In some examples, the radiopaque anchor marker is located at the connector distal end portion.

In some examples, the radiopaque plunger marker is located at the plunger proximal end portion.

In some examples, the radiopaque anchor marker and the radiopaque plunger marker are axially continuous with each other in the distally-biased position of the plunger.

According to some aspects of the disclosure, there is provided a method comprising advancing a tissue perforation apparatus to a target tissue over a guidewire, wherein the tissue perforation apparatus comprises an anchor device and a needle. The anchor device comprises a radiopaque anchor marker configured to move along with helical anchor of the anchor device, and a radiopaque plunger marker configured to move along with a plunger of the anchor device.

In some examples, the method further comprises advancing the helical anchor towards a target tissue to anchor the anchor device to the target tissue.

In some examples, the method further comprises determining that a distance between the radiopaque anchor marker and the radiopaque plunger marker has changed.

In some examples, the method further comprises, upon determining that the distance between the radiopaque anchor marker and the radiopaque plunger marker has changed, advancing the needle to form a pilot opening at the target tissue.

In some examples, the determining that the distance has changed comprises determining that the distance between the radiopaque anchor marker and the radiopaque plunger marker is greater after the advancing the helical anchor than before the advancing the helical anchor.

In some examples, the radiopaque anchor marker and the radiopaque plunger marker form a continuous radiopaque image under fluoroscopy, prior to the advancing the helical anchor.

In some examples, the method further comprising, before the advancing the helical anchor, maintaining the plunger in a distally-biased position relative to the helical anchor.

In some examples, the anchoring the helical anchor comprises compressing the biasing member.

In some examples, the advancing the helical anchor to anchor the anchor device comprises rotating the anchor device in a first rotational direction.

According to some aspects of the disclosure, there is provided a method comprising advancing a tissue perforation apparatus to a target tissue over a guidewire, wherein the tissue perforation apparatus comprises an anchor device and a needle. The guidewire extends through an anchor shaft and a helical anchor distal to the anchor shaft of the anchor device.

In some examples, the method further comprises advancing the helical anchor towards a target tissue to anchor the anchor device to the target tissue.

In some examples, the method further comprises distally advancing the guidewire through an anchor channel of the helical anchor.

In some examples, the method further comprises determining that the guidewire does not bend upon contact with the target tissue.

In some examples, the method further comprises, upon determining that the guidewire does not bend, advancing the needle to form a pilot opening at the target tissue.

In some examples, the determining that the guidewire does not bend comprises determining that the guidewire is not bent radially away from the helical anchor.

In some examples, the determining that the guidewire does not bend comprises determining that the guidewire does not exit the helical anchor.

In some examples, a method or device can include any of the features recited in Examples 1-151 below.

The aspects of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the invention will become more apparent from the following detailed description, which proceeds with reference to the accompanying figures.

### BRIEF DESCRIPTION OF THE FIGURES

Some examples of the invention are described herein with reference to the accompanying figures. The description, together with the figures, makes apparent to a person having ordinary skill in the art how some examples may be practiced. The figures are for the purpose of illustrative description and no attempt is made to show structural details of an example in more detail than is necessary for a fundamental understanding of the invention. For the sake of clarity, some objects depicted in the figures are not to scale.

In the Figures:

FIG. 1 is a sectional view of an aortic root.

FIG. 2A shows a cross-sectional view of a prosthetic heart valve implanted in the native aortic valve of within the aortic root of FIG. 1, according to an example.

FIG. 2B shows the implanted prosthetic heart valve of FIG. 1A as viewed from the ascending aorta, according to an example.

FIG. 3 shows a valve-in-valve implantation within the native aortic valve of FIG. 1, according to an example.

FIG. 4 illustrates an exemplary tissue perforation apparatus that includes an anchor device and a needle.

FIG. 5 shows a perspective sectional view of a distal portion of an exemplary tissue perforation apparatus.

FIG. 6A is a cross-sectional view of a distal portion of an exemplary tissue perforation apparatus with a needle shown in a first position, proximal to a target tissue.

FIG. 6B is a cross-sectional view of the tissue perforation apparatus of FIG. 6A, with the needle shown in a second position, extending through the target tissue.

FIG. 7 shows an exemplary tissue modification system, comprising a steerable delivery apparatus and the tissue perforation apparatus extending therethrough.

FIGS. 8A-8B illustrate exemplary steps in a method for anchoring an exemplary helical anchor to a host leaflet of an existing valvular structure.

FIG. 9A shows an unbent configuration of a guidewire pushed through a helical anchor which is successfully anchored to the leaflet.

FIG. 9B shows a bent configuration of a guidewire pushed through a helical anchor which is not anchored to the leaflet.

FIGS. 10A-10D illustrate exemplary steps in a method for creating a pilot puncture in the leaflet.

FIG. 11A shows an exemplary dilation apparatus of the system extending through the steerable delivery apparatus.

FIG. 11B shows an exemplary dilation apparatus of the system extending through a delivery catheter that extends through the steerable delivery apparatus.

FIGS. 12A-12E illustrate exemplary steps in a method for forming a leaflet opening.

FIG. 12F is a simplified side view of a guest prosthetic valve in a crimped configuration positioned inside the leaflet opening formed in the existing valvular structure.

FIG. 12G is a simplified side view of the guest prosthetic valve of FIG. 12F expanded inside the existing valvular structure.

FIG. 13A shows the hole-dilation balloon positioned within a pilot puncture of the host leaflet in a deflated state.

FIG. 13B shows the hole-dilation balloon of FIG. 13A inflated within the host leaflet.

FIG. 13C shows the guest prosthetic valve positioned in the leaflet opening after removal of the hole-dilating balloon of FIG. 11B.

FIG. 14A is a perspective view of a host prosthetic valve subsequent to forming a leaflet opening thereof.

FIG. 14B is a perspective view of a guest prosthetic valve expanded within a leaflet opening of a host prosthetic valve.

FIG. 15A is a cross-sectional view of a distal end portion of an exemplary tissue perforation apparatus that includes a plunger disposed around the helical anchor.

FIG. 15B is an enlarged view of a region of FIG. 15A that includes an anchor marker and a plunger marker of the anchor device.

FIGS. 16A-16C illustrate steps in a method for utilizing the tissue perforation apparatus of FIGS. 15A-15B to confirm proper anchoring of the helical anchor to the leaflet.

### DETAILED DESCRIPTION

For purposes of this description, certain aspects, advantages, and novel features of the examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present, or problems be solved. The technologies from any example can be combined with the technologies described in any one or more of the other examples. In view of the many possible examples to which the principles of the disclosed technology may be applied, it should be recognized that the illustrated examples are only preferred examples and should not be taken as limiting the scope of the disclosed technology.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

All features described herein are independent of one another and, except where structurally impossible, can be used in combination with any other feature described herein.

As used in this application and in the claims, the singular forms “a”, “an”, and “the” include the plural forms unless the context clearly dictates otherwise. Additionally, the terms “have” or “includes” means “comprises”. Further, the terms “coupled”, “connected”, and “attached”, as used herein, are interchangeable and generally mean physically, mechanically, chemically, magnetically, and/or electrically coupled or linked and does not exclude the presence of intermediate elements between the coupled or associated items absent specific contrary language. As used herein, “and/or” means “and” or “or”, as well as “and” and “or”.

Directions and other relative references may be used to facilitate discussion of the drawings and principles herein, but are not intended to be limiting. For example, certain terms may be used such as “inner”, “outer”, “upper”, “lower”, “inside”, “outside”, “top”, “bottom”, “interior”, “exterior”, “left”, right”, and the like. Such terms are used, where applicable, to provide some clarity of description when dealing with relative relationships, particularly with respect to the illustrated examples. Such terms are not, however, intended to imply absolute relationships, positions, and/or orientations. For example, with respect to an object, an “upper” part can become a “lower” part simply by turning the object over. Nevertheless, it is still the same part and the object remains the same.

The term “plurality” or “plural” when used together with an element means two or more of the element. Directions and other relative references (for example, inner and outer, upper and lower, above and below, left and right, and proximal and distal) may be used to facilitate discussion of the drawings and principles herein but are not intended to be limiting.

The terms “proximal” and “distal” are defined relative to the use position of a delivery apparatus. In general, the end of the delivery apparatus closest to the user of the apparatus is the proximal end, and the end of the delivery apparatus farthest from the user (for example, the end that is inserted into a patient's body) is the distal end. The term “proximal” when used with two spatially separated positions or parts of an object can be understood to mean closer to or oriented towards the proximal end of the delivery apparatus. The term “distal” when used with two spatially separated positions or parts of an object can be understood to mean closer to or oriented towards the distal end of the delivery apparatus. The terms “longitudinal” and “axial” are interchangeable, and refer to an axis extending in the proximal and distal directions, unless otherwise expressly defined.

The terms “axial direction”, “radial direction”, and “circumferential direction” have been used herein to describe the arrangement and assembly of components relative to the geometry of the frame of the prosthetic valve, or the geometry of an inflatable balloon that can be used to expand a prosthetic valve. Such terms have been used for convenient description, but the disclosed examples are not strictly limited to the description. In particular, where a component or action is described relative to a particular direction, directions parallel to the specified direction as well as minor deviations therefrom are included. Thus, a description of a component extending along an axial direction of an apparatus disclosed herein or a shaft thereof does not require the component to be aligned with a center of the shaft; rather, the component can extend substantially along a direction parallel to a central axis of the apparatus or a shaft thereof.

As used herein, the terms “integrally formed” and “unitary” refer to a construction that does not include any welds, fasteners, or other means for securing separately formed pieces of material to each other.

As used herein, operations that occur “simultaneously” or “concurrently” occur generally at the same time as one another, although delays in the occurrence of operation relative to the other due to, for example, spacing between components, are expressly within the scope of the above terms, absent specific contrary language.

As used herein, terms such as “first”, “second”, and the like are intended to serve as respective labels of distinct components, steps, etc. and are not intended to connote or imply a specific sequence or priority. For example, unless otherwise stated, a step of performing a second action and/or of forming a second component may be performed prior to a step of performing a first action and/or of forming a first component.

As used herein, the term “substantially” means the listed value and/or property and any value and/or property that is at least 75% of the listed value and/or property. Equivalently, the term “substantially” means the listed value and/or property and any value and/or property that differs from the listed value and/or property by at most 25%. For example, “at least substantially parallel” refers to directions that are fully parallel, and to directions that diverge by up to 22.5 degrees.

In the present disclosure, a reference numeral that includes an alphabetic label (for example, “a”, “b”, “c”, etc.) is to be understood as labeling a particular example of the structure or component corresponding to the reference numeral. Accordingly, it is to be understood that components sharing like names and/or like reference numerals (for example, with different alphabetic labels or without alphabetic labels) may share any properties and/or characteristics as disclosed herein even when certain such components are not specifically described and/or addressed herein.

Throughout the figures of the drawings, different superscripts for the same reference numerals are used to denote different examples of the same elements. Examples of the disclosed devices and systems may include any combination of different examples of the same elements. Specifically, any reference to an element without a superscript may refer to any alternative example of the same element denoted with a superscript. In order to avoid undue clutter from having too many reference numbers and lead lines on a particular drawing, some components will be introduced via one or more drawings and not explicitly identified in every subsequent drawing that contains that component.

Described herein are devices and methods for puncturing a tissue, such as a leaflet, which can be performed as part of a procedure for implanting prosthetic valves and modifying leaflets of an existing valvular structure in a patient's heart. Prior to or during implantation of the prosthetic heart valve within the existing valvular structure, a tissue piercing assembly that includes a needle can be provided in the ascending aorta of a patient and can be used to pierce, lacerate, slice, tear, cut or otherwise modify a leaflet or commissure of the existing valvular structure. In some examples, the existing valvular structure can be a native aortic valve (for example, normal or abnormal, such as bicuspid aortic valve (BAV)) or a prosthetic valve previously implanted in the native aortic valve. The modification can avoid, or at least reduce the likelihood of, issues that leaflets of the existing valvular structure might otherwise cause once the prosthetic heart valve has been fully installed, for example, obstruction of blood flow to the coronary arteries, improper mounting due to a non-circular valve cross-section, and/or restricted access to the coronary arteries if subsequent intervention is required. While described with respect to aortic valve, it should be understood that the disclosed examples can be adapted to deliver devices that can modify existing valvular structure, and in some implementations, implant prosthetic devices, to and/or in any of the native annuluses of the heart (for example, the aortic, pulmonary, mitral, and tricuspid annuluses), and can be used with any of various delivery approaches (for example, retrograde, antegrade, transseptal, transventricular, transatrial, etc.).

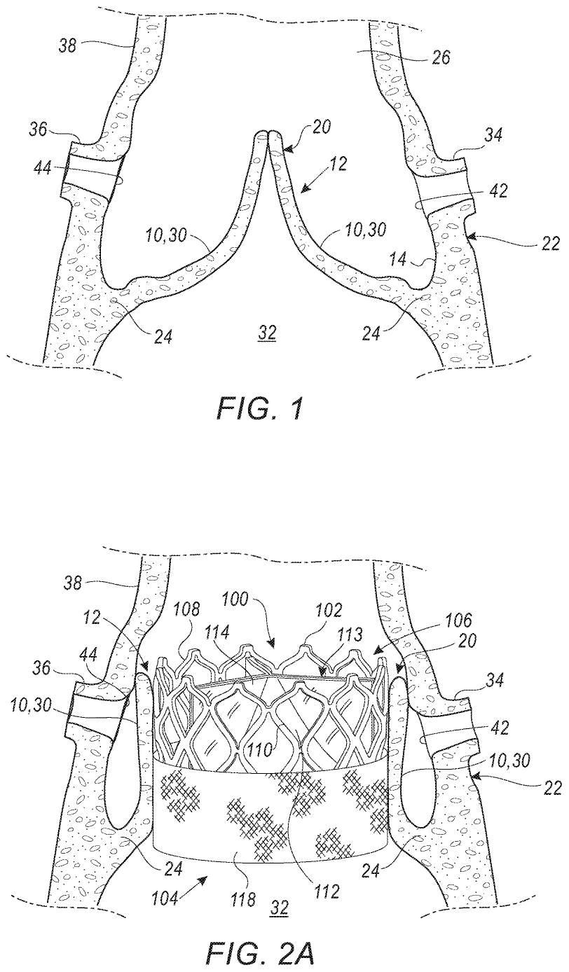

FIG. 1 illustrates an anatomy of the aortic root 22, which is positioned between the left ventricle 32 and the ascending aorta 26. The aortic root 22 includes a native aortic valve 20 having a native valvular structure 29 comprising a plurality of native leaflets 30. Normally, the native aortic valve 20 has three leaflets (only two leaflets are visible in the simplified illustration of FIG. 1), but aortic valves with fewer than three leaflets are possible. The leaflets 30 are supported at native commissures by the aortic annulus 24, which is a ring of fibrous tissue at the transition point between the left ventricle 32 and the aortic root 22. The leaflets 30 can cycle between open and closed positions (the closed position is shown in FIG. 1) to regulate flow of blood from the left ventricle 32 to the ascending aorta 26. Branching off the aortic root 22 are the coronary arteries 34, 36. The coronary artery ostia 42, 44 are the openings that connect the aortic root 22 to the coronary arteries 34, 36.

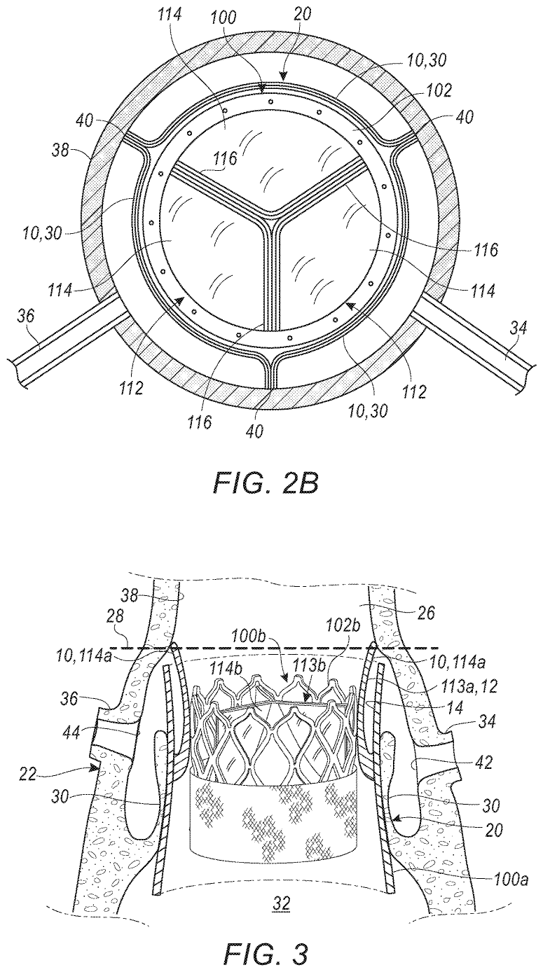

FIGS. 2A-2B show an exemplary prosthetic valve 100 that can be implanted in a native heart valve, such as the native aortic valve 20 of FIG. 1. The term “prosthetic valve”, as used herein, refers to any type of a prosthetic valve deliverable to a patient's target site over a catheter, which is radially expandable and compressible between a radially compressed, or crimped, state, and a radially expanded state. Thus, the prosthetic valve can be crimped on or retained by an implant delivery apparatus (not shown) in the radially compressed state during delivery, and then expanded to the radially expanded state once the prosthetic valve reaches the implantation site. The expanded state may include a range of diameters to which the valve may expand, between the compressed state and a maximal diameter reached at a fully expanded state. Thus, a plurality of partially expanded states may relate to any expansion diameter between radially compressed or crimped state, and maximally expanded state. A prosthetic valve of the current disclosure (for example, prosthetic valve 100) may include any prosthetic valve configured to be mounted within the native aortic valve, the native mitral valve, the native pulmonary valve, and the native tricuspid valve.

It is understood that the prosthetic valves disclosed herein may be used with a variety of implant delivery apparatuses. Balloon expandable valves generally involve a procedure of inflating a balloon within a prosthetic valve, thereby expanding the prosthetic valve within the desired implantation site. Once the valve is sufficiently expanded, the balloon is deflated and retrieved along with a delivery apparatus (not shown). Self-expandable valves include a frame that is shape-set to automatically expand as soon an outer retaining shaft or capsule (not shown) is withdrawn proximally relative to the prosthetic valve. Mechanically expandable valves are a category of prosthetic valves that rely on a mechanical actuation mechanism for expansion. The mechanical actuation mechanism usually includes a plurality of expansion and locking assemblies (such as the prosthetic valves described in U.S. Pat. No. 10,603,165, International Application No. PCT/US 2021/052745 and U.S. Provisional Application Nos. 63/85,947 and 63/209904, each of which is incorporated herein by reference in its entirety), releasably coupled to respective actuation assemblies of a delivery apparatus, controlled via a handle (not shown) for actuating the expansion and locking assemblies to expand the prosthetic valve to a desired diameter. The expansion and locking assemblies may optionally lock the valve's diameter to prevent undesired recompression thereof, and disconnection of the actuation assemblies from the expansion and locking assemblies, to enable retrieval of the delivery apparatus once the prosthetic valve is properly positioned at the desired site of implantation.

FIGS. 2A-2B show an example of a prosthetic valve 100, which can be a balloon expandable valve or any other type of valve, illustrated in an expanded state. The prosthetic valve 100 can comprise an outflow end 106 and an inflow end 104. In some instances, the outflow end 106 is the proximal end of the prosthetic valve 100, and the inflow end 104 is the distal end of the prosthetic valve 100. Alternatively, depending for example on the delivery approach of the valve, the outflow end can be the distal end of the prosthetic valve, and the inflow end can be the proximal end of the prosthetic valve.

The term “outflow”, as used herein, refers to a region of the prosthetic valve through which the blood flows through and out of the prosthetic valve 100.

The term “inflow”, as used herein, refers to a region of the prosthetic valve through which the blood flows into the prosthetic valve 100.

In the context of the present application, the terms “lower” and “upper” are used interchangeably with the terms “inflow” and “outflow”, respectively. Thus, for example, the lower end of the prosthetic valve is its inflow end and the upper end of the prosthetic valve is its outflow end.

In the context of the present application, the terms “lower” and “upper” are used interchangeably with the terms “distal to” and “proximal to”, respectively. Thus, for example, a lowermost component can refer to a distal-most component, and an uppermost component can similarly refer to a proximal-most component.

The terms “longitudinal” and “axial”, as used herein, refer to an axis extending in the proximal and distal directions, unless otherwise expressly defined.

The prosthetic valve 100 comprises an annular frame 102 movable between a radially compressed configuration and a radially expanded configuration, and a valvular structure 113 that comprises prosthetic valve leaflets 114 mounted within the frame 102. The frame 102 can be made of various suitable materials, including plastically-deformable materials such as, but not limited to, stainless steel, a nickel based alloy (for example, a nickel-cobalt-chromium alloy such as MP35N alloy), polymers, or combinations thereof. When constructed of a plastically-deformable materials, the frame 102 can be crimped to a radially compressed state on a balloon catheter, and then expanded inside a patient by an inflatable balloon or equivalent expansion mechanism. Alternatively or additionally, the frame 102 can be made of shape-memory materials such as, but not limited to, nickel titanium alloy (for example, Nitinol). When constructed of a shape-memory material, the frame 102 can be crimped to a radially compressed state and restrained in the compressed state by insertion into a shaft or equivalent mechanism of a delivery apparatus.

In the example illustrated in FIGS. 2A-2B, the frame 102 is an annular, stent-like structure comprising a plurality of intersecting struts 108. In this application, the term “strut” encompasses axial struts, angled struts, laterally extendable struts, commissure windows, commissure support struts, support posts, and any similar structures described by U.S. Pat. Nos. 7,993,394 and 9,393,110, which are incorporated herein by reference. A strut 108 may be any elongated member or portion of the frame 102. The frame 102 can include a plurality of strut rungs that can collectively define one or more rows of cells 110. The frame 102 can have a cylindrical or substantially cylindrical shape having a constant diameter from the inflow end 104 to the outflow end 106 as shown, or the frame can vary in diameter along the height of the frame, as disclosed in U.S. Pat. No. 9,155,619, which is incorporated herein by reference.

The struts 108 can include a plurality of angled struts and vertical or axial struts. At least some of the struts 108 can be pivotable or bendable relative to each other, so as to permit frame expansion or compression. For example, the frame 102 can be formed from a single piece of material, such as a metal tube, via various processes such as, but not limited to, laser cutting, electroforming, and/or physical vapor deposition, while retaining the ability to collapse/expand radially in the absence of hinges and like.

A valvular structure 113 of the prosthetic valve 100 can include a plurality of prosthetic valve leaflets 114 (for example, three leaflets), positioned at least partially within the frame 102, and configured to regulate flow of blood through the prosthetic valve 100 from the inflow end 104 to the outflow end 106. While three leaflets 114 arranged to collapse in a tricuspid arrangement, are shown in the example illustrated in FIGS. 2A-2B, it will be clear that a prosthetic valve 100 can include any other number of leaflets 114. Adjacent leaflets 114 can be arranged together to form prosthetic valve commissures 116 that are coupled (directly or indirectly) to respective portions of the frame 102, thereby securing at least a portion of the valvular structure 113 to the frame 102. The prosthetic valve leaflets 114 can be made from, in whole or part, biological material (for example, pericardium), bio-compatible synthetic materials, or other such materials. Further details regarding transcatheter prosthetic valves, including the manner in which leaflets 114 can be coupled to the frame 102 of the prosthetic valve 100, can be found, for example, in U.S. Pat. Nos. 6,730,118, 7,393,360, 7,510,575, 7,993,394, 8,652,202, and 11,135,56, all of which are incorporated herein by reference in their entireties.

In some examples, the prosthetic valve 100 can comprise at least one skirt or sealing member. For example, the prosthetic valve 100 can include an inner skirt (not shown in FIG. 2A-2B), which can be secured to the inner surface of the frame 102. Such an inner skirt can be configured to function, for example, as a sealing member to prevent or decrease perivalvular leakage. An inner skirt can further function as an anchoring region for leaflets 114 to the frame 102, and/or function to protect the leaflets 114 against damage which may be caused by contact with the frame 102, for example during valve crimping or during working cycles of the prosthetic valve 100. An inner skirt can be disposed around and attached to the inner surface of frame 102, while the leaflets can be sutured to the inner skirt along a scalloped line (not shown). An inner skirt can be coupled to the frame 102 via sutures or another form of coupler.

The prosthetic valve 100 can comprise, in some examples, an outer skirt 118 mounted on the outer surface of frame 102 (as shown in FIGS. 2A-2B), configured to function, for example, as a sealing member retained between the frame 102 and the surrounding tissue of the native annulus against which the prosthetic valve is mounted, or against an inner side of a previously implanted valve in the case of ViV procedures (described further below), thereby reducing risk of paravalvular leakage (PVL) past the prosthetic valve 100. The outer skirt 118 can be coupled to the frame 102 via sutures or another form of coupler.

Any of the inner skirt and/or outer skirt can be made of various suitable biocompatible materials, such as, but not limited to, various synthetic materials (for example, PET) or natural tissue (for example pericardial tissue). In some cases, the inner skirt can be formed of a single sheet of material that extends continuously around the inner surface of frame 102. In some cases, the outer skirt 118 can be formed of a single sheet of material that extends continuously around the outer surface of frame 102.

The cells 110, defined by interconnected struts 108, define cell openings 112. While some of the cell openings 112 can be covered by the inner skirt and/or the outer skirt, at least a portion of the cell opening 112 can remain uncovered, such as cell openings 112 which are closer to the outflow end 106 of the prosthetic valve.

FIGS. 2A-2B illustrate a hypothetical coronary artery obstruction that could occur in some cases from implantation of a prosthetic valve 100 within the native aortic valve 20. In this example, the prosthetic valve 100 is the guest valve or new valve, and the native aortic valve 20 is the host valve or old valve.

During implantation of the prosthetic valve 100, the prosthetic valve 100 is positioned within a central region defined between the native leaflets 30, which are also the host leaflets 10 for the example illustrated in FIG. 2A-2B. The prosthetic valve 100 is then radially expanded against the host leaflets 10. As illustrated, the host leaflets 10 form a tube around the frame 102 of the prosthetic valve 100 after the prosthetic valve 100 is radially expanded to the working diameter. As further illustrated, expansion of the prosthetic valve 100 displaces the host leaflets 10 outwards towards the coronary ostia 42, 44 such that the host leaflets 10 contact a portion of the aortic root 22 surrounding the coronary ostia 42, 44, causing coronary artery obstruction.

For an existing implanted prosthetic valve, the valvular structure may naturally degrade over time thereby requiring repair or replacement in order to maintain adequate heart functions. In a Valve-in-Valve (ViV) procedure, a new prosthetic heart valve is mounted within the existing, degrading prosthetic heart valve in order to restore proper function. FIG. 3 illustrates an exemplary hypothetical coronary artery obstruction that could occur in some cases from implantation of a prosthetic valve 100b within a previously implanted prosthetic valve 100a (for example, after a ViV procedure). In this example, the prosthetic valve 100b is the guest valve or new valve, and the prosthetic valve 100a is the host valve or old valve. In this example, the prosthetic valve 100a was previously implanted within the orifice of the native aortic valve 20. Each of the prosthetic valves 100a, 100b can have the general structure of the prosthetic valve 100 described with reference to FIGS. 2A-2B, though in some examples, each of the prosthetic valves 100a, 100b can be a different type of prosthetic valve. For example, a balloon expandable guest valve 100b can be implanted inside a previously implanted mechanically expandable or self-expandable host valve 100a.

During implantation of the prosthetic valve 100b, the prosthetic valve 100b is positioned within a central region defined between the leaflets 114a of the prosthetic valve 100a, which now take the role of host leaflet 10. The prosthetic valve 100b is then radially expanded against the host leaflets 10 (i.e., against the prosthetic valve leaflets 114c). As illustrated, the radial expansion of the prosthetic valve 100a results in outward displacement of the host leaflets 10. As further illustrated, the host leaflets 10 are displaced such that the host leaflets 10 contact the aortic root 22 at positions superior to the coronary artery ostia 42, 44, causing coronary artery ostia obstruction. Alternatively, the guest prosthetic valve 100b can displace the host leaflets 114a outwardly against the frame 102a of the host valve 100a, thereby blocking the flow of blood through the frame 102a to the coronary ostia 42, 44.

In some patient anatomies (for example, when the outflow end 106 of the prosthetic valve 100 is at the STJ level 28 and the diameter of the prosthetic valve 100 is similar to the STJ diameter such that the frame 102 touches or is very close to the aortic wall 38 at the STJ level 28), the host leaflets 10 may compromise the ability for future access into the coronary arteries 34, 36 or perfusion through the frame 102 to the coronary arteries 34, 36 during the diastole phase of the cardiac cycle. Similar problems may occur in some patient anatomies either when a guest prosthetic valve 100b is percutaneously expanded within a previously implanted host prosthetic valve 100a, or when a prosthetic valve 100 is percutaneously expanded within a native valve, displacing the native leaflets 30 outward toward the coronary ostia 42, 44.

The risk illustrated in FIG. 3 may be higher when the host valve is a bioprosthetic valve without a frame or when the leaflets of the host valve are external to a frame. Risk of coronary artery ostia obstruction can increase in a cramped aortic root or when the coronary artery ostium sits low. In the examples illustrated in FIGS. 2A-3, the host leaflets 10 are shown obstructing both coronary artery ostia 42, 44. In some cases, only one host leaflet 10 may obstruct a respective coronary artery ostium. For example, the risk of obstructing the left coronary ostium 42 tends to be greater than obstructing the right coronary ostium 44 because the left coronary ostium 42 typically sits lower than the right coronary ostium 44.

The term “host valve” as used herein refers to a native heart valve in which a prosthetic valve is implanted or a previously implanted prosthetic valve in which a new prosthetic valve is implanted. Moreover, in any of the examples disclosed herein, when the host valve is a previously implanted prosthetic valve, the host valve can be a surgically implanted prosthetic heart valve (known as a “surgical valve”) or a transcatheter heart valve. The term “guest valve”, as used herein, refers to a prosthetic valve implanted in a host valve, which can be either a native heart valve or a previously implanted prosthetic valve. Similarly, the term “host leaflets 10”, as used herein, refers to native leaflets 30 of a native valve in which a new guest prosthetic valve 100 is implanted, or to prosthetic valve leaflets 114a of a previously implanted host valve 100a in which a new guest prosthetic valve 100b is implanted.

When a guest prosthetic valve 100 is deployed inside a host valvular structure 12, it displaces the host leaflets 10 of the host valve radially outwards, towards and against a host interior surface 14, which can be the interior surface of the aortic wall 38 if the host valve is the native valve, or an interior surface of the frame 102a of a previously implanted prosthetic valve 100a serving as the host valve.

To avoid obstruction of blood flow to the coronary arteries 34, 36, the valvular structure 12 of the existing host valve (whether a native aortic valve or a previously implanted prosthetic valve) can be modified by components of a delivery apparatus prior to or during implantation of a new prosthetic valve within the existing valvular structure 12. In some examples, the host valvular structure 12 is modified by piercing, lacerating, tearing, slicing, and/or cutting one or more host leaflets 10 (for example, a free end of the host leaflet 10 or a commissure of adjacent host leaflets 10, which can be a native commissure 40 for a native aortic valve 20, or a prosthetic valve commissure 116 for a previously implanted host prosthetic valve 100) using the delivery apparatus. The modification thus disrupts the impermeable tubular structure that would otherwise be formed by the existing host leaflets 10, thereby allowing blood to flow to the coronary arteries 34, 36.

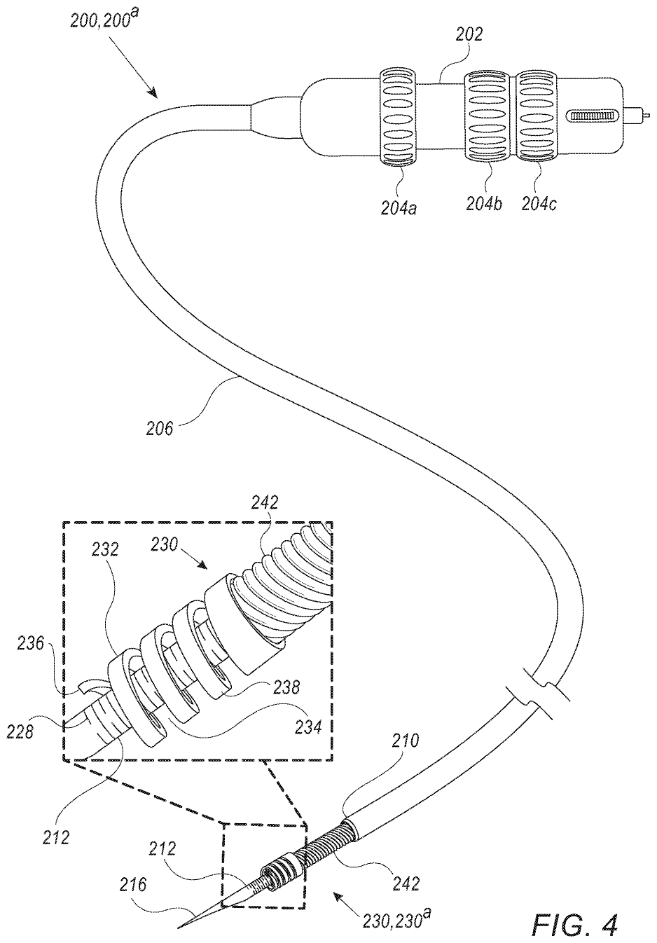

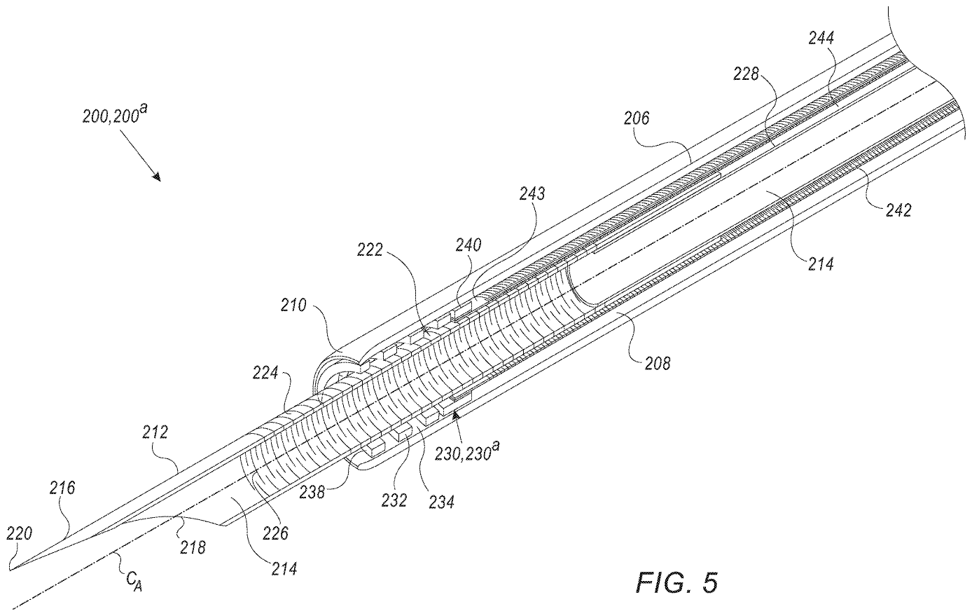

FIG. 4 illustrates an exemplary tissue perforation apparatus 200, which can include a delivery catheter 206 attached to a handle 202 and extending distally therefrom. FIG. 5 shows a perspective sectional view of a distal portion of the tissue penetration apparatus of FIG. 4. The delivery catheter 206 defines a delivery catheter lumen 208 and can have a delivery catheter distal portion 210 which can be, in some examples, an atraumatic distal end portion 210, such as by being rounded and/or being curved radially inwards, or otherwise formed to include an outer surface tapering in the distal direction.

The tissue perforation apparatus 200 can include a hollow needle 212 and an anchor device 230 through which the needle 212 can extend. The needle 212 comprises a needle head 216 and a needle shaft 222 extending proximally from the needle head 216, collectively defining a needle lumen 214. The needle head 216 is configured to pierce a target tissue, such as a host leaflet 10 of a host valvular structure 12, to form a pilot puncture 50 in the host leaflet 10. The needle head 216 can define an angled surface 218 terminating at a sharp needle tip 220 configured to facilitate piercing the host leaflet 10 when the needle 212 is pressed thereagainst.

In some examples, at least a portion of the needle shaft 222 comprises slits arranged in a desired pattern, such as that of known hypo-tubes, to enhance flexibility thereof. In the example illustrated in FIGS. 4-5, at least part of the needle shaft 222, such as a distal portion 224 thereof, is shown to include a plurality of circumferential slits 226 axially spaced from each other, so as to form circumferential bands separating between adjacent circumferential slits 226, with axially extending connecting portions connecting adjacent bands. Two adjacent circumferential bands can be connected by a plurality of angularly spaced connecting portions defined between ends of corresponding circumferential slits 226. In such arrangements, the slitted part of the needle shaft 222, such as the distal portion 224, exhibits sufficient flexibility to allow it to flex as it is pushed through a tortuous pathway without kinking or buckling, and/or to bend when passed through bent portions of the vasculature and/or through bends of a catheter is extends through.

While a specific pattern is illustrated in FIGS. 4-5, which can be a laser cut pattern, it is to be understood that the pattern of slits 226 and/or the size of the slits 226 and/or axial distances between the slits 226 can vary along the length of the corresponding slitted part of the needle shaft 222 in order to vary stiffness of the slitted part of the needle shaft 222 along its length. For example, the axial distance between adjacent slits 226 can decrease from the proximal end to the distal end of the slitted part of the needle shaft 222 to provide greater stiffness near the proximal end and greater flexibility near the distal end of the needle shaft 222.

In some examples, the slitted part of the needle shaft 222 can extend along the entire length of the needle shaft 222 or at least a significant portion of a length thereof. In some examples, the needle shaft 222 can include a distal portion 224 that includes slits, such as slits 226, and a proximal portion 228 extending proximally from the distal portion 224, which can be devoid of slits, as illustrated in FIG. 5. In some examples, the distal portion 224 and the proximal portion 228 of the needle shaft 222 are separate components that can be affixed to each other, and may be made from similar or different materials. For example, a laser-cut metallic tube that includes slits 226 can be used to form the distal portion 224, while the proximal portion 228 can be made of a polymeric material. The distal portion 224 can allow for increased flexibility along a distal part of the needle 212, allowing it to be steered towards a target tissue, such as a host leaflet 10, to improve precision of positioning and penetration, while the polymeric proximal portion 228, devoid of such slits, may be less flexible than the distal portion 224, yet flexible enough to allow it to passively bend along curved portions of the patient's vasculature, for example.

In some examples, the distal portion 224 extends along less than 50% of the length of the entire needle shaft 222. In some examples, the distal portion 224 extends along less than 30% of the length of the entire needle shaft 222. In some examples, the distal portion 224 extends along less than 25% of the length of the entire needle shaft 222. In some examples, the distal portion 224 extends along less than 20% of the length of the entire needle shaft 222. In some examples, the needle head 216 can be continuous with and/or integrally formed with the distal portion 224. For example, when the distal portion 224 is formed from a metallic tube, the needle head 216 can be an integral extension of the tube, together forming a unitary component, while the needle head 216 can be devoid of slits.

In some implementations of a needle 212, the length of the needle shaft 222 extending through a patient's vasculature, all the way to a host leaflet 10, such as a leaflet in an aortic valve, can be in the order of more than 2 meters, such as between 2-3 meters or even longer. Laser cutting metallic tubes have such lengths can be costly. Limiting the distal portion 224 of the needle shaft 222 to be formed as a hypotube, while the optionally longer proximal portion 228 is made of a polymeric material, can advantageously reduce manufacturing costs. The proximal portion 228 can be affixed, at its distal end, to a proximal end of the distal portion 224, by any method known in the art such as gluing, overmolding, and the like.

While the needle shaft 222 is shown in the example illustrated in FIG. 5 to be formed of a distal portion 224 that includes slits 226, and a proximal portion 228 devoid of slits, it is to be understood any exemplary needle 212 disclosed herein can be, in some examples, slitted along its entire length, such as by being made of a metallic laser-cut hypotube that defines the entirety of the needle shaft 222.

The anchor device 230 of apparatus 200 includes a helical anchor 232 which is attached, directly or via one or more intermediate components, to an anchor shaft 242. The helical anchor 232 defines an anchor channel 234 and has a sharp anchor tip 236 configured to allow it to engage and penetrate a target tissue, such as a host leaflet 10 of a host valvular structure. The helical anchor 232 can be used in combination with the needle 212 which can extend through the anchor channel 234 towards and through a host leaflet 10, for modifying the host leaflet 10. The anchor shaft 242 can extend through the delivery catheter lumen 208. In some examples, the delivery catheter 206 and the anchor shaft 242 can be configured to be axially movable relative to each other. For example, a distally oriented movement of the anchor shaft 242 relative to the delivery catheter 206 can expose the helical anchor 232 from the delivery catheter 206. The terms “helical anchor 232” and “anchor 232”, as used herein, are interchangeable.

The anchor shaft 242 can be a torque shaft, configured to be movable rotatably relative to a central axis C_{A} thereof and/or rotatable relative to another shaft of the apparatus 200, such as relative to the delivery catheter 206. The helical anchor 232 is affixed, directly or via one or more intermediate components, to the anchor shaft 242, such that rotation of the anchor shaft 242 effects rotation of the helical anchor 232 therewith. The anchor shaft 242 defines a lumen 244 which is in fluid communication with the anchor channel 234. In some examples, at least a portion of the anchor shaft 242 is formed as a hypotube, configured to increase flexibility thereof. In some examples, at least a portion of the anchor shaft 242 comprises a helical hollow strand (HHS) tube.

In some examples, the apparatus 200 can include a handle 202, wherein the proximal ends of the delivery catheter 206, the anchor shaft 242 and/or the needle shaft 212 can be coupled to the handle 202. During delivery through the patient's vasculature, the handle 202 can be maneuvered by an operator (for example, a clinician or a surgeon) to axially advance or retract components of the apparatus 200, such as the delivery catheter 206, the anchor shaft 242 and/or the needle shaft 222.

The handle 202 can include a shaft-rotating mechanism which can be optionally operable by a knob of the handle, such as the rotatable knob 204b. A proximal end of the anchor shaft 242 can be operatively connected to a manually rotatable shaft-rotating mechanism or a motorized shaft-rotating mechanism that allows the operator (such as a clinician) to effect rotation of the anchor shaft 242 and the helical anchor 232.

The handle can include additional adjustment mechanisms controllable by additional knobs to maneuver additional components of the apparatus 200, such as axial movement of a needle 212. The terms “tissue perforation apparatus 200” and “apparatus 200”, as used herein, are interchangeable.

In some examples, a helical anchor 232 can be a tube-cut anchor. Manufacturing of a tube-cut helical anchor 232 can employ any suitable cutting method, such as, but not limited to, laser cutting, water-jet cutting, plasma cutting, and the like. The helical anchor 232 can define one or more helical turns 238 continuously extending between the helical anchor proximal end 240 and the anchor tip 236. In some examples, the helical anchor can be formed from a rounded wire shaped to form the helical turns of the anchor 232. Sharpening the anchor tip 236 can employ grinding or any other suitable sharpening method.

Various exemplary implementations for apparatus 200 and/or components thereof can be referred to, throughout the specification, with superscripts, for ease of explanation of features that refer to such exemplary implementations. It is to be understood, however, that any reference to structural or functional features of any apparatus, device or component, without a superscript, refers to these features being commonly shared by all specific exemplary implementations that can be also indicated by superscripts. In contrast, features emphasized with respect to an exemplary implementation of any apparatus, device or component, referred to with a superscript, may be optionally shared by some but not necessarily all other exemplary implementations. For example, apparatus 200^{a} and anchor device 230^{a} thereof, illustrated in FIGS. 4-5, are exemplary implementations of respective apparatus 200 and anchor device 230, and thus can include any of the features described for apparatus 200 and anchor device 230 throughout the current disclosure, except that the helical anchor 232 of anchor device 230^{a} can be directly attached, for example at the helical anchor proximal end 240, to a distal end 243 of the anchor shaft 243.

In some cases, it may be desirable to prevent the needle head 216 from extending past the helical anchor 232 while forming a puncture through a target tissue, such as a host leaflet 10, to protect the anatomical structures in the vicinity of host leaflet 10 from being engaged or punctured by the needle sharp tip 220. In some examples, the length of helical anchor 232 is selected such that after being anchored to host leaflet 10, a sufficient length of the anchor 232 extends distally from the leaflet 10 to allow extension of the needle 212 through the anchor channel 234 and leaflet 10 during formation of a pilot puncture, without the needle head 216 extending beyond the anchor tip 236.

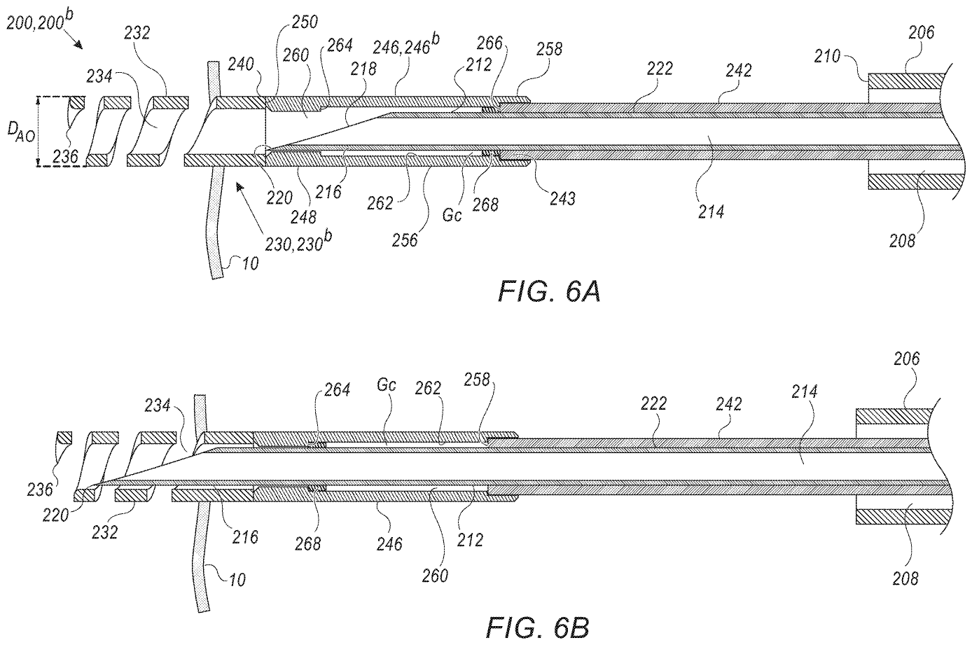

FIGS. 6A-6B are cross-sectional views of a distal end portion of an exemplary apparatus 200^{b}. Apparatus 200^{b} and anchor device 230^{b} thereof are exemplary implementations of apparatus 200 and anchor device 230, and thus can include any of the features described for apparatus 200 and anchor device 230 throughout the current disclosure, except that the anchor device 230^{b} of apparatus 200^{b} further includes a connector 246 defining the chamber 260, extending between the helical anchor 232 and the anchor shaft 242. The connector 246 and its chamber 260 can serve as a movement limiting segment configured to prevent axial movement of the needle head 216 pas a certain axial position relative to the anchor 232, such as beyond the anchor tip 236.

A needle 212 of apparatus 200^{b} can axially move between a first position and a second position relative the helical anchor 232, wherein the first position is proximal to the second position, and wherein, in the second position, the needle head 216 extends through the anchor channel 234, without extending past the anchor tip 236. FIG. 6A shows the needle 212 in the first position, and FIG. 6B shows the needle 212 in the second position.

In some examples, connector 246 can be attached to, or integrally formed with, the anchor shaft 242. In some examples, connector 246 can be attached to, or integrally formed with, the helical anchor 232. The connector 246 can be affixed at its distal end portion 248 to the helical anchor 232, and can be affixed at its proximal end portion 258 to the anchor shaft 242. The chamber 260 can define a chamber inner surface 262 having a diameter that is greater than the inner diameter of the anchor shaft 242. A connector intermediate portion 256 can be defined between the connector distal end portion 248 and the connector proximal end portion 258.

The chamber 260 is continuous with the anchor shaft lumen 244 and the anchor channel 234, allowing axial movement of the needle 212 therethrough. The connector 246 further includes a chamber distal step 164, extending radially inwards at a distal end of the inner chamber 260. In some examples, the anchor shaft 242 can terminate at a distal end 243 that defines a proximal step 266 of the chamber 260. For example, the thickness of the anchor shaft 242 can define a proximal step 266 of the chamber 260 at the transition from the anchor shaft distal end 243 to the chamber inner surface 262.

The needle can include a stopper 268. In some examples, the stopper 268 can be implemented as an outer protrusion, which is axially movable within the chamber 260. In the example illustrated in FIGS. 6A-6B, the outer protrusion 268 extends radially outwards from the needle shaft 222, and optionally from the distal portion 224 of the needle shaft, configured to move along the chamber 260 inside connector 246. When the needle 212 extends through the connector 246, the chamber 260 defines a radial gap G_{C} between the outer surface of the needle shaft 222 and the chamber inner surface 262, into which the outer protrusion 268 extends.

The outer protrusion 268 can be in the form of a ring disposed around the circumference of the needle shaft 222, in the form of a protrusion that does not necessarily circles around the entire circumference of the needle shaft 222, or in the form of a series of protrusions that can be equally or unequally spaced from each other around the circumference of the needle shaft 222. The outer protrusion 268 can be integrally formed with the needle shaft 222, or provided as a separate component affixed to the needle shaft 222, such as by welding, adhering, or any other suitable manner of attachment known in the art.

The helical anchor 232 is shown in FIG. 6A to be already passed through the target tissue, such as host leaflet 10, while the needle head 216 is proximal to the helical anchor 232 in the first position, such that the needle tip 220 is proximal to the anchor channel 234. The needle head 216 can be entirely concealed inside connector 246 while the outer protrusion 268 is proximal to the chamber distal step 264. As shown in FIG. 6B, distal movement of the needle 212 is allowed up to a maximal exposed length of the needle 212 through the anchor channel 234, at which point the outer protrusion 268 reaches and contacts the chamber distal step 264, thereby preventing further distal advancement of the needle 212 relative to the anchor 232.

While the chamber distal step 264 is illustrated in FIGS. 6A-6B to be formed by a narrowing in diameter of the connector 246, it is to be understood that this is shown by way of illustration and not limitation, and that any of the chamber distal step 264 and/or chamber proximal step 266 can be formed by a protrusion extending radially inwards from an inner wall of the connector 246, without necessarily spanning the entire circumference around a central axis C_{A} of the anchor device 230.

While the exemplary anchor device 230^{b} is shown in FIGS. 6A-6B to include a chamber proximal step 266, it is to be understood that in some examples, the anchor device 230^{b} can include only a chamber distal step 264 without a chamber proximal step, so as to limit distal advancement of the needle 212 relative to the anchor 232, yet allow undisturbed proximal movement of the needle 212.

In some examples, the needle shaft proximal portion 228 can extend into the handle 202 and be attached to a mechanism configured to facilitate axial movement of the needle 212 relative to the anchor device 230, optionally controllable by a knob such as rotatable knob 204c. The anchor shaft 242 is rotatable around its axis C_{A}, while the needle 212 is not rotatable around the axis C_{A}. In use, upon approaching the host leaflet 10, the anchor device 230 can be rotated to screw the helical anchor 232 into the host leaflet 10, after which the needle 212 can be advanced to puncture the leaflet 10 without extending beyond a predetermined length, to protect anatomical structures at the site of treatment.

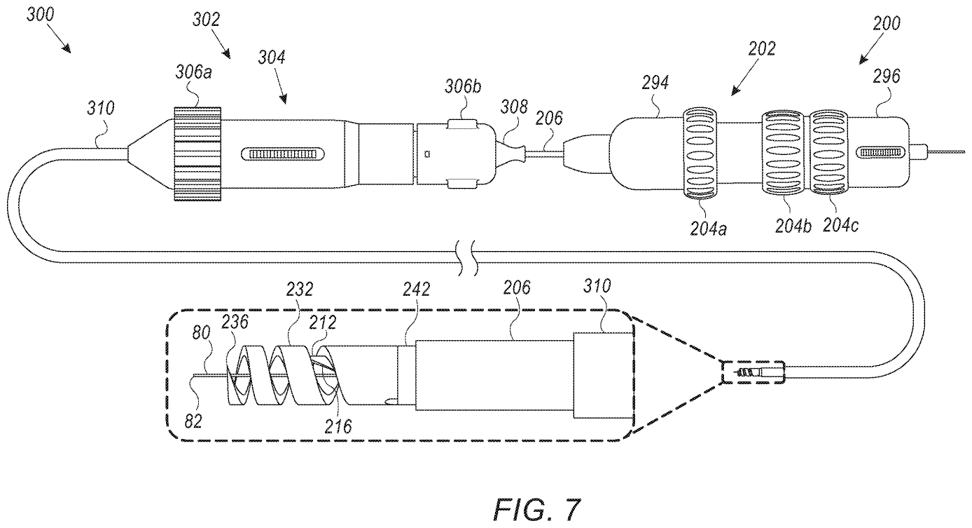

In some examples, the tissue perforation apparatus 200 can be used as part of a tissue modification system 300. FIG. 7 shows an exemplary tissue modification system 300, which can include a steerable delivery apparatus 302. A steerable delivery apparatus 302 can include an outer catheter 310, optionally implemented as a steerable catheter. The steerable outer catheter 310 can be advanced towards the valvular structure 12 over a guidewire 80, and the delivery catheter 206 can be passed, along with the anchor device 230 and needle 212, through the outer catheter 310, over the guidewire 80, towards the host leaflet 10.

In some examples, the steerable delivery apparatus 302 can include a handle 304, wherein the outer catheter 310 can extend distally from the handle 304. The handle 304 can be maneuvered to control the outer catheter 310. In some examples, the handle 304 can include a steering mechanism configured to adjust the curvature of the distal end portion of the outer catheter 310. In the illustrated example, the handle 304 can include an adjustment member, such as the illustrated rotatable knob 306a, which in turn is operatively coupled to the proximal end portion of a pull wire (not shown). The pull wire can extend distally from the handle 304 through the outer catheter 310 and has a distal end portion affixed to the outer catheter 310 at or near the distal end of the outer catheter 310. Rotating the knob 306a can increase or decrease the tension in the pull wire, thereby adjusting the curvature of the distal end portion of the outer catheter 310. Further details on steering or flex mechanisms for the delivery apparatus can be found in U.S. Pat. No. 9,339,384, which is incorporated by reference herein. The terms “tissue modification system 300” and “system 300”, as used herein, are interchangeable.

The steerable outer catheter 310 can be advanced through the patient's vasculature towards the target site of treatment, optionally without the tissue perforation apparatus 200, taking advantage of the steerability of the outer catheter 310 to navigate it during delivery, after which the delivery catheter 206 can be inserted through a rear port 308 of the handle 304 and advanced through the outer catheter 310.

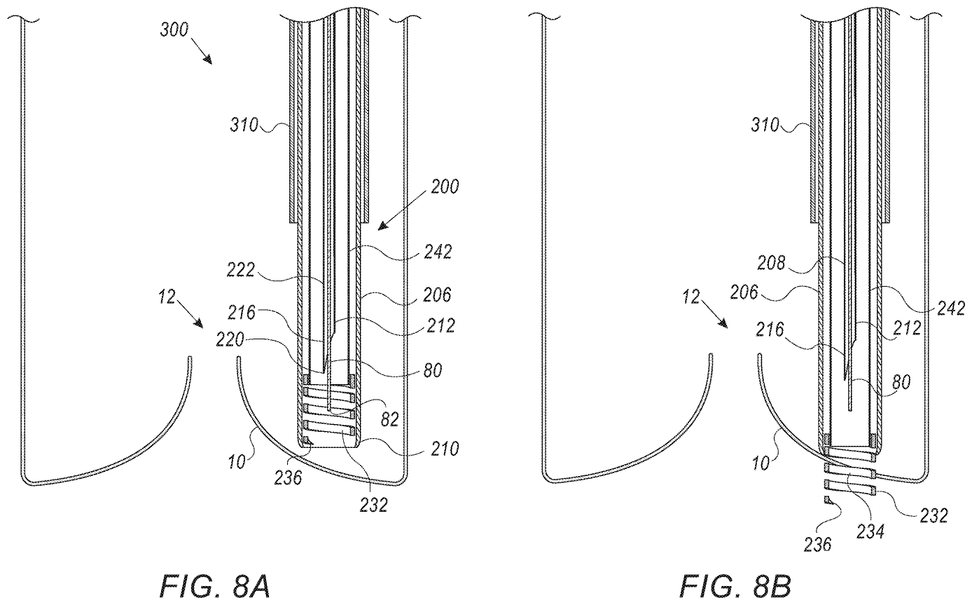

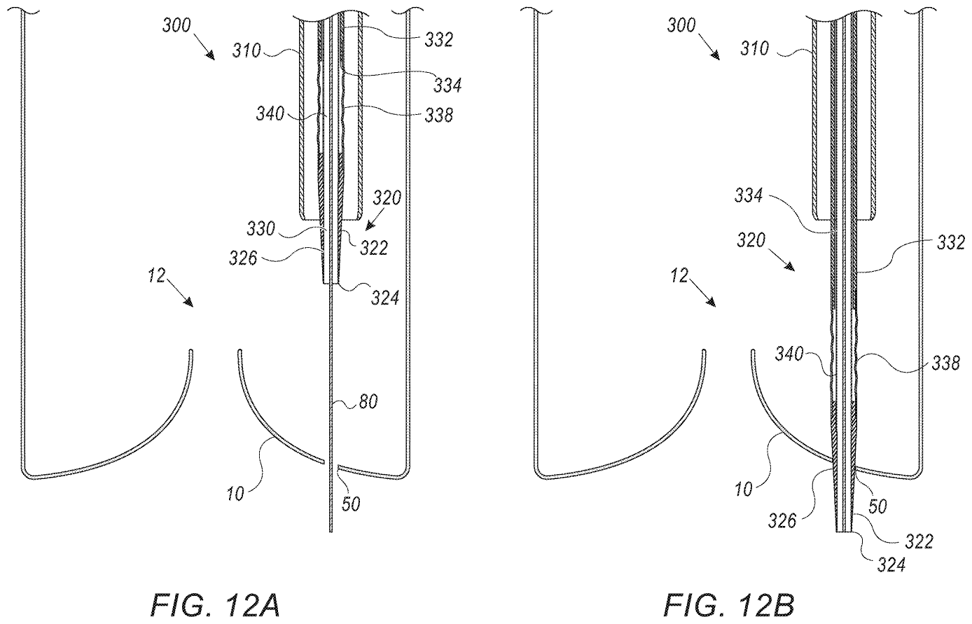

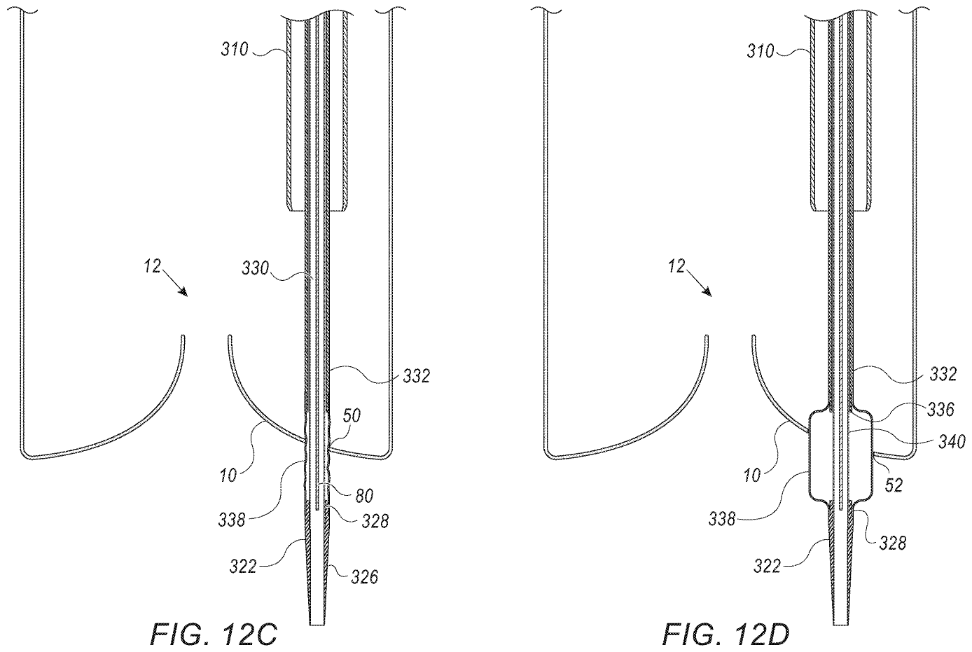

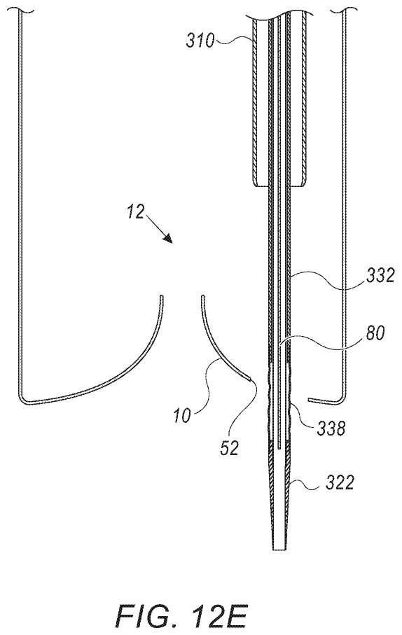

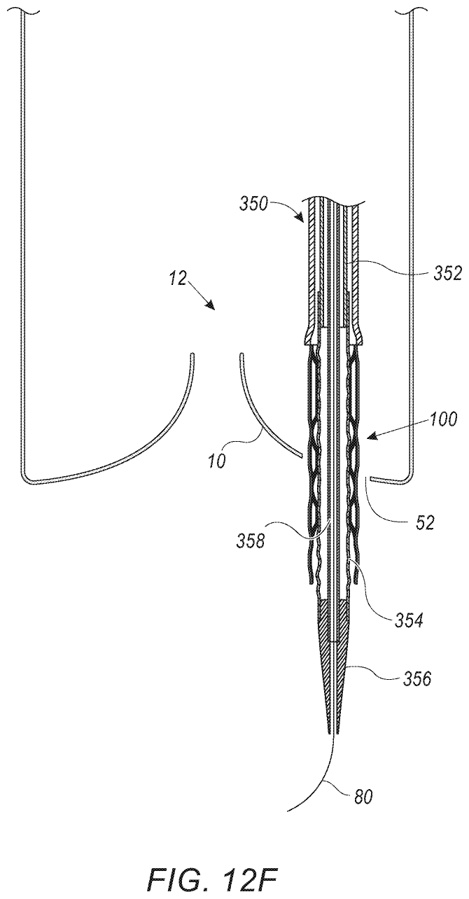

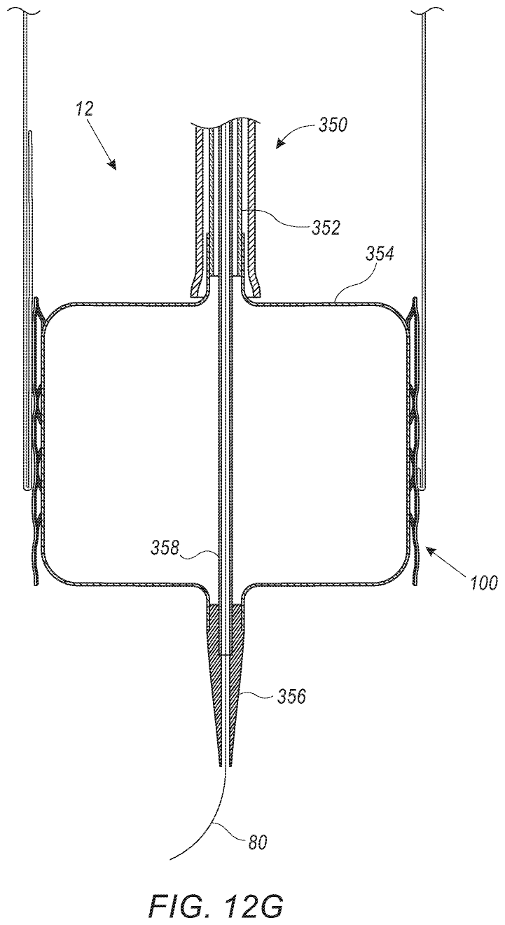

FIGS. 8A-8B illustrate some steps in a method for utilizing a tissue perforation apparatus 200, optionally extendable through an outer catheter 310 of a system 300, for securing a helical anchor 232 to a target tissue. These steps can be part of a method for forming an opening within a target tissue. An exemplary implementation of the method is illustrated in FIGS. 8A-8B, 10A-10D and 12A-12E with respect to forming a leaflet opening inside a host leaflet, which can be performed prior to implanting a guest prosthetic valve inside the host valvular structure, as further described below with respect to FIGS. 12F-12G for example. The apparatus 200 can be used to perforate a host leaflet 10, such as a native leaflet 30 or a prosthetic valve leaflet 114 of a previously implanted prosthetic valve.

The distal end portion of the apparatus 200, which can include an atraumatic distal end portion 210 of the delivery catheter 206, is configured to be advanced towards the host leaflet 10, optionally through a pre-inserted steerable outer catheter 310. Positioning the delivery catheter distal end portion 210 relative to the host leaflet 10 may comprise advancing the delivery catheter 206 toward the leaflet over the guidewire 80. The needle 212 can be configured to accommodate a guidewire 80 that can extend through the needle lumen 214.

During delivery, the helical anchor 232 can be retained inside delivery catheter lumen 208, such that the anchor tip 236 is at or proximal to the delivery catheter distal end portion 210, as illustrated in FIG. 8A. This position conceals the sharp tip 236 of the helical anchor 232 from the surrounding anatomy, to protect the anatomical structures, as well as the outer catheter 310 through which it can be advanced, from being engaged or punctured by the anchor tip 236 during advancement towards the site of treatment. The needle head 216 can be similarly retained inside the anchor device 230, such that the needle tip 220 is at or proximal to the anchor tip 236. This position conceals the sharp tip 220 of the needle head 216 from the surrounding anatomy and/or outer catheter 310, to similarly protect them from being engaged or punctured by the needle sharp tip 220 during advancement towards the site of treatment.

The anchor 232 can be approximated to the host leaflet 10 and rotated in a first rotational direction (such as clockwise or counterclockwise) around its axis C_{A}, causing it to engage and penetrate the host leaflet 10, thereby securing the helical anchor 232 to host leaflet 10 as shown in FIG. 8B. The tissue material of host leaflet 10 can be retained between successive helical turns 238 of the anchor 232. In some examples, the delivery shaft distal end portion 210 can be in contact with, and/or slightly pushed against, the host leaflet 10, prior to rotating the helical anchor 232 to screw it into the host leaflet 10, which can stretch and/or flatten the host leaflet 10 to some extent along a plane perpendicular to the axial direction of anchor device 230 and needle 212 advancement, which can increase stability of the leaflet for improved engagement with the helical anchor 232 at it is being screwed thereinto.

Following engagement of the anchor 232 with the host leaflet 10, the needle 212 should be advanced to puncture the tissue material. However, prior to needle advancement, verification of proper engagement of the helical anchor 232 with the host leaflet 10 is required. If the helical anchor 232 did not penetrate and pass through the leaflet 10 following rotation thereof, and remains, for example, proximal to the leaflet surface, advancement of the needle may either entirely miss the leaflet, cause the needle head to slightly slide along the leaflet prior to penetration such that the penetration position occurs elsewhere along the leaflet, or may contact and damage nearby anatomical structures. Thus, it is desirable to confirm that the helical anchor 232 is properly anchored the host leaflet 10 prior to advancement of the needle 212 therethrough.

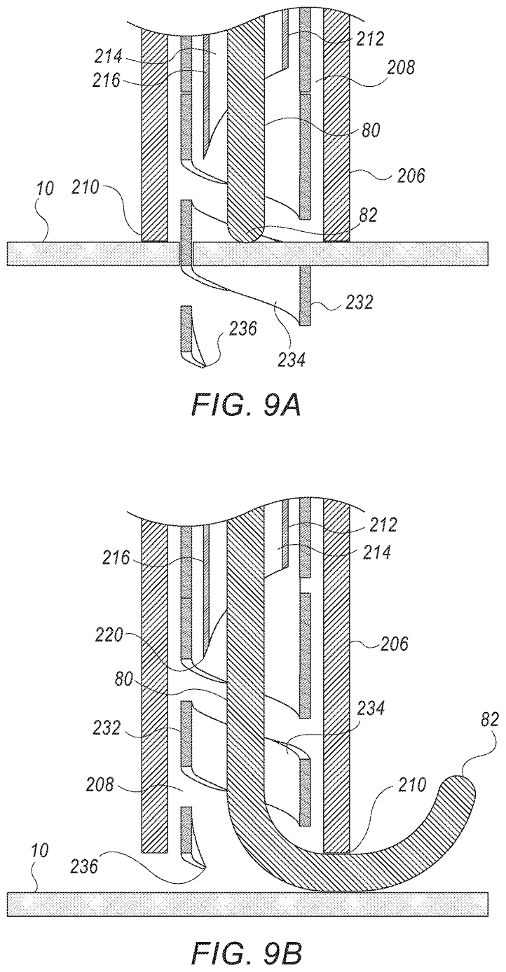

FIGS. 9A and 9B show one exemplary method for confirming anchoring of the helical anchor to the host leaflet 10. FIG. 9A shows one optional scenario of proper penetration of the helical anchor 232 through the host leaflet 10, following the stage described above with respect to FIG. 8B. Anchoring confirmation can be performed under fluoroscopy or other radiation-based imaging technique, configured to produce relatively bright images of radiopaque components of the apparatus or system. While leaflets are not necessarily detectable under fluoroscopy, a guidewire 80 that can be formed of or include metallic materials or otherwise marked by radiopaque coating or markings, so that it can be visually detected in real-time on a fluoroscopy screen or another imaging technique. In order to verify anchor penetration, the guidewire 80 can be distally advanced through the anchor channel 234, and the shape of a portion of the guidewire 80 distal to the anchor shaft 242 (e.g., the portion of the guidewire 80 shown in FIGS. 9A-9B) can be tracked under fluoroscopy (or other adequate imaging modality).

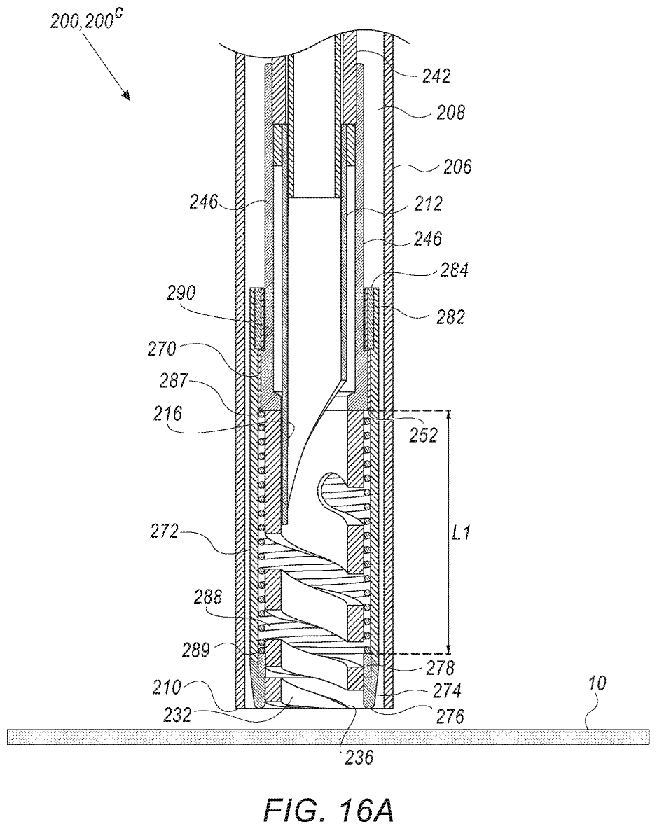

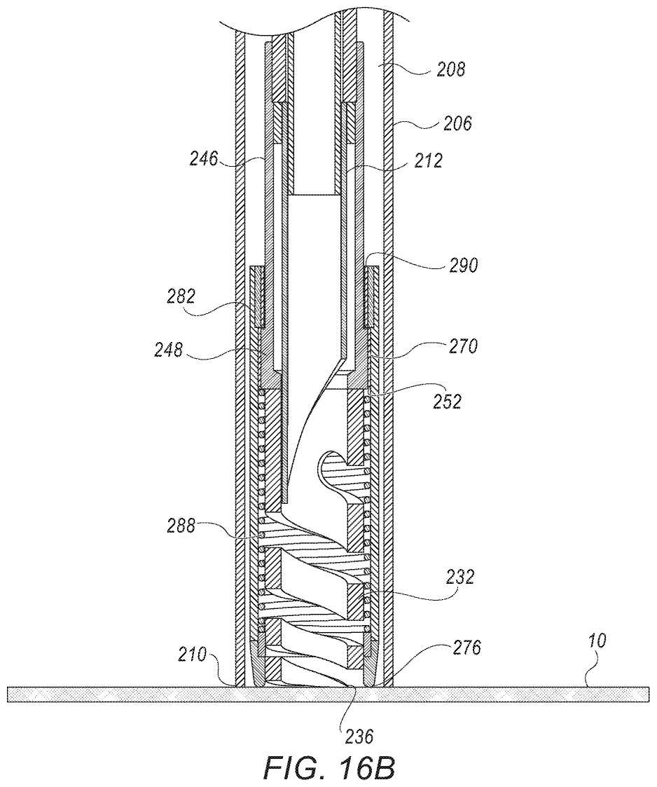

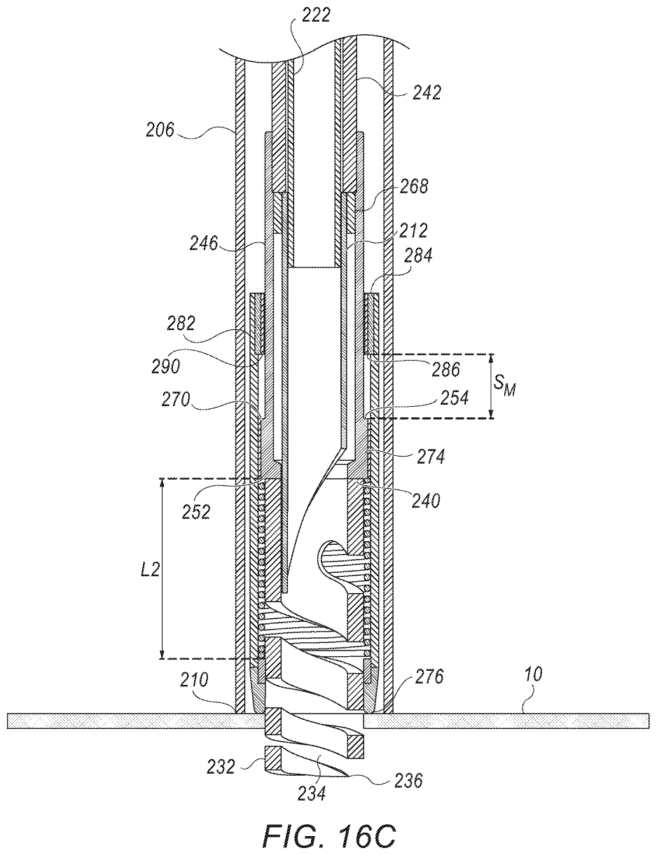

While most conventional guidewires are radiopaque, if a guidewire used to navigate apparatus 200 is not made of radiopaque materials, it can include radiopaque marking along at least a distal portion thereof (e.g., the portion of the guidewire 80 shown in FIGS. 9A-9B). In some examples, a guidewire radiopaque marking can be formed by means of radiopaque inks and adhesives, and applied on the corresponding guidewire (for example as at least along a distal portion thereof) in a number of ways, such as screen printing, high speed roller printing, coating, dipping, etc. In some examples, any of the radiopaque anchor markers described herein (e.g., see radiopaque anchor markers 270, 290 shown in FIGS. 16A-16C) can be separately formed components, such as annular rings or bands that are mounted on or inside the corresponding anchor device components. Radiopaque materials of a guidewire radiopaque marking can include, but are not limited to, gold, platinum, tantalum, tungsten alloy, platinum iridium alloy, palladium, barium sulfate, bismuth, and/or any other material which is opaque to fluoroscopy, X-rays, or similar radiation or any combination thereof.

In some examples, a radiopaque marking of the guidewire can have a higher radiopacity than one or more other components of the tissue perforation apparatus 200, such as helical anchor 232. In some examples, a radiopaque marking of the guidewire can have a higher radiopacity than the needle 212. In some examples, a radiopaque marking of the guidewire can have a higher radiopacity than the delivery catheter 206.

In some examples, the guidewire 80 extends through the needle lumen 214. However, since the needle head 216 is positioned proximal to the host leaflet 10 prior to advancement of the needle 212 to form a puncture in the leaflet, the guidewire 80 can extend past the needle tip 220 and be exposed out of the needle lumen 214 as it approaches the leaflet 10. When the guidewire tip 82 contacts the host leaflet 10, further advancement of the guidewire 80 is no longer possible. In some examples, continued push force applied to the guidewire 80 can cause a portion of the guidewire 80 to bend sideways along the surface of the leaflet 10, for example, as shown in FIG. 9B. In the state shown in FIG. 9A, when the helical anchor 232 is passed through or anchored to the leaflet 10, a portion of the guidewire 80 extending beyond the needle lumen 214 is still retained within the anchor channel 234, such that sideways-oriented movement past the inner walls of the helical anchor 232 may be prohibited. Thus, when the helical anchor 232 is anchored to the leaflet 10, the guidewire 80 will be forced to retain a relatively straight configuration in the axial direction inside the portion of the helical anchor 232 extending proximally from the leaflet 10.

FIG. 9B shows an alternative scenario in which the helical anchor 232 is not anchored to the host leaflet 10. In this example, advancement of the guidewire 80 can bend the guidewire 80 (e.g., sideways) when the guidewire tip 82 contacts the leaflet 10. In some examples, the delivery catheter distal end 210 and the anchor tip 236 can be proximally offset from the host leaflet 10 prior to and/or during guidewire 80 advancement, allowing a distal end portion of the guidewire 80 to easily bend sideways through the gap between the delivery catheter 208, as well as the anchor 232, and the leaflet 10.

In some examples, the anchor tip 236 can be in close proximity to, or even in contact with, the leaflet 10, without penetrating through the leaflet 10, in which case, when the tip 82 of the guidewire 80 is pushed against the leaflet 10, it can displace the leaflet 10 distally away from the anchor 232 to form a gap through which the guidewire can slide sideways between the anchor 232 and the leaflet 10.

In some examples, the delivery catheter distal end 210 can be in close proximity to, or even in contact with, the leaflet 10 (similar to the relative position of the delivery catheter 210 illustrated in FIG. 9A, for example), in which case, when the guidewire 80 is pushed against the leaflet 10, it can similarly displace the leaflet 10 distally away from the delivery catheter 206 to form a gap through which the guidewire can slide sideways between the delivery catheter distal end 210 and the leaflet 10.

Thus, according to the method described above, after rotating the anchor device 230 to screw the helical anchor 232 through a host leaflet 10, the guidewire 80 can be distally advanced and tracked under fluoroscopy, wherein a relatively straight configuration of the distal portion of the guidewire 80 (e.g., a portion of the guidewire 80 extending at least partially through the helical anchor 232) may be indicative of proper penetration of the anchor 232 through the leaflet 10, while a bent configuration of a distal portion of the guidewire, which can extend, for example, beyond the diameter of the helical anchor 232, may be indicative of failed penetration, in which case the guidewire 80 can be partially retracted and an additional attempt can be made to screw the anchor into the host leaflet 10.

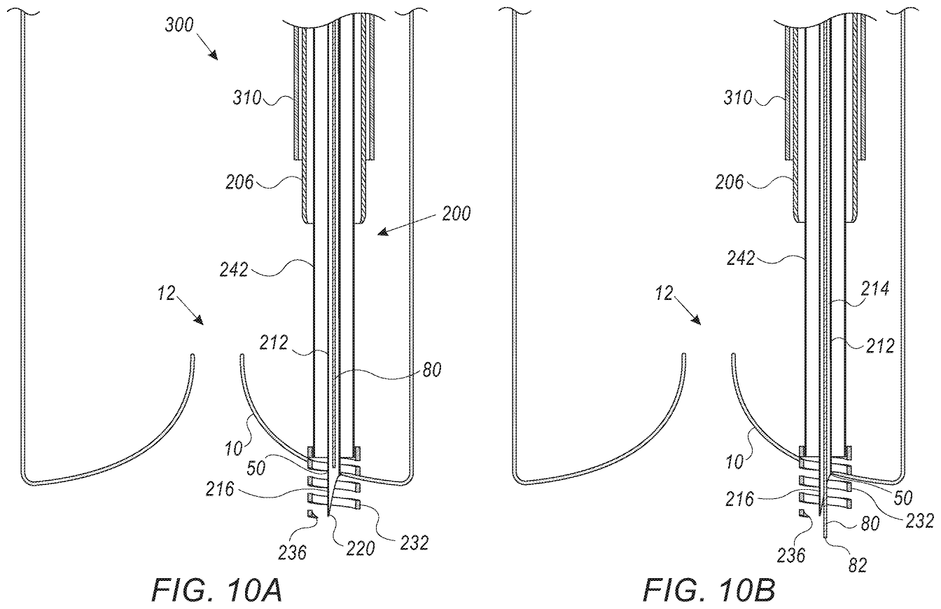

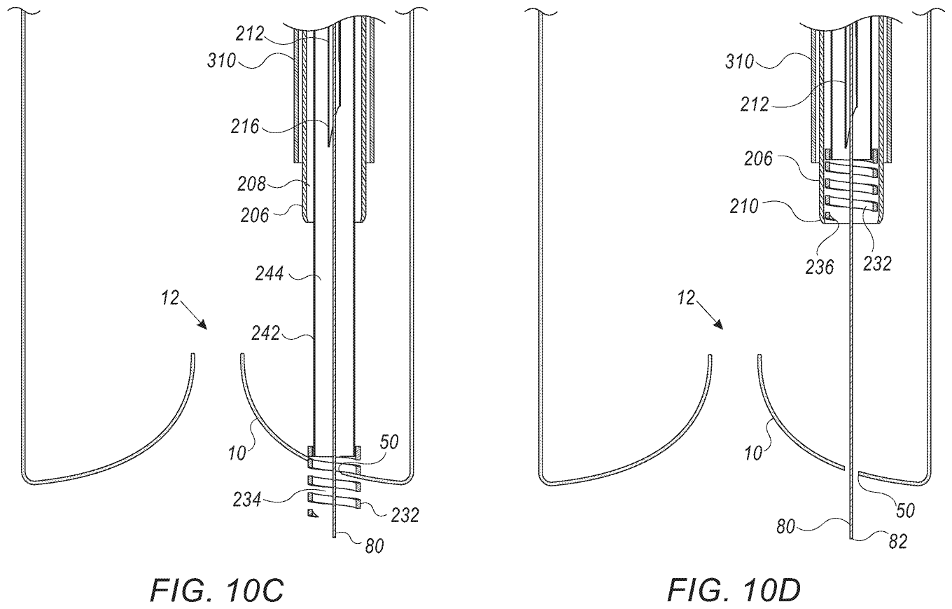

FIGS. 10A-10D illustrate some follow-up steps in a method for utilizing a tissue perforation apparatus 200 for forming pilot puncture within a target tissue, such as a host leaflet 10, subsequent to securing the helical anchor 232 to the host leaflet 10, and optional verification of adequate engagement of the anchor 232 with the leaflet 10, such as described, for example, with respect to FIG. 9A. At this stage, as shown in FIG. 10A, the needle 212 can be distally advanced to puncture the host leaflet 10 to form a pilot puncture 50 within host leaflet 10, for example when its needle head 216 is axially translated relative to anchor device 230.

An attempt to pass a needle 212 through a relatively thin and movable tissue component, such as a leaflet, in the absence of an anchor, might push the leaflet to some extent prior to eventually penetrating therethrough, which, even if achieving the goal of eventually puncturing the leaflet, might result in a wrong or somewhat offset position of the puncture hole due to this undesired relative movement. Advantageously, the helical anchor 232 captures the host leaflet 10 and stabilizes it during formation of a pilot puncture 50 by a needle 212 being pushed against and through the host leaflet 10.

Once the needle head 216 is positioned, at least partially, past the host leaflet 10, the guidewire 80 can be advanced through the needle lumen 214 to terminate with guidewire tip 82 at a position distal to the pilot puncture 50 of host leaflet 10 as shown in FIG. 10B.

Subsequent to forming the pilot puncture 50 and optionally advancing the guidewire 80 to extend therethrough, the needle 212 can be optionally retracted, as shown in FIG. 10C, and the anchor device 230 can be rotated in a second rotational direction, opposite to the first rotational direction, so as to release the helical anchor 232 from the host leaflet 10, which can be similarly retracted by being then axially pulled away from the host leaflet 10, as shown in FIG. 10D, leaving the guidewire 80 extending through the pilot puncture 50.

It is to be understood that the order of procedural steps described above with respect to FIGS. 10B-10D is merely shown for illustrative purpose, and that in some examples, reverse rotation of the helical anchor 232 to release it from the host leaflet 10 and retract it can be performed prior to needle 212 retraction. In some examples, counter-rotation of the helical anchor 232 to release it from the host leaflet 10 can be performed prior to needle 212 retraction, and axial retraction of the helical anchor 232 can be performed subsequent to needle 212 retraction. In some examples, needle 212 retraction can be performed simultaneously with counter-rotation of the helical anchor 232 to release it from the host leaflet 10 and/or axial retraction of the helical anchor 232 from the host leaflet.

In some examples, the guidewire 80 can be advanced simultaneously with advancement of the needle 212 during formation of the pilot puncture 50. In some examples, the guidewire 80 can be advanced to terminate distal to the host leaflet 10 after formation of the pilot puncture 50 by the needle 212, as illustrated in FIG. 10B. In some examples, the guidewire 80 can be advanced through pilot puncture 50 to terminate distal to the host leaflet 10 after retrieval of the needle 212, optionally prior to release of the anchor 232 from the host leaflet 10.

In some examples, advancement of the guidewire 80 to position the guidewire tip 82 distal to the pilot puncture 50 can be performed subsequent to counter-rotation of the helical anchor 232 to release it from the host leaflet 10 and/or axial retraction of the helical anchor 232, while the needle 212 is still positioned inside of pilot puncture 50, after which the needle 212 can be retracted. In some examples, advancement of the guidewire 80 to position the guidewire tip 82 distal to the pilot puncture 50 can be performed after needle 212 retraction while the helical anchor 232 is still engaged with the host leaflet 10, after which the anchor 232 can be released and retracted.

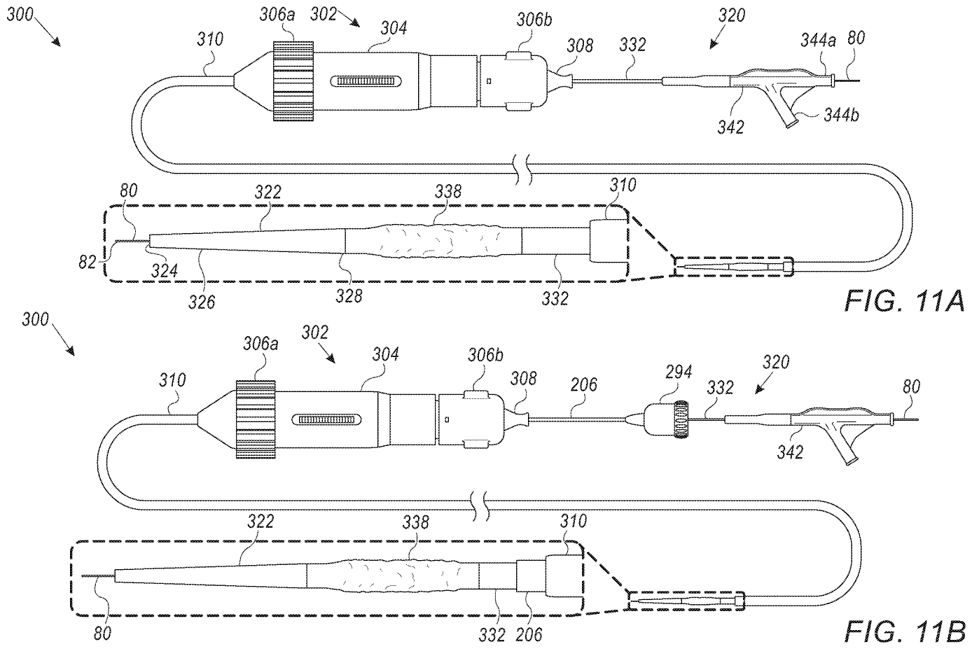

In some examples, a tissue modification system 300 can further include a dilation apparatus 320, having a hole-dilating balloon 338 mounted on a balloon catheter 332, as shown in FIG. 11A for example. After formation of the pilot puncture 50, the needle 212 and the anchor device 230 can be retrieved from the patient's body, optionally by retraction through the outer catheter 310 and out of the handle 304 of steerable delivery apparatus 302, while leaving the outer catheter 310 in position and the guidewire 80 extending through the pilot puncture 50.

In some examples, the tissue perforation apparatus 200 can be completely removed from the system 300 after formation of the pilot puncture 50, such that the needle 212 and the anchor device 230 are retracted along with the delivery catheter 210 out of the patient's body and out of the handle 304 of steerable delivery apparatus 302, after which the balloon catheter 332 of dilation apparatus 320 can be inserted, optionally through the rear port 308 of the handle 304, through the handle 304 and the outer catheter 310, as shown in FIG. 11A. In some examples, the balloon catheter 332 can be advanced through the outer catheter 310, optionally over the guidewire 80, towards the host leaflet 10, as shown for example in FIG. 12A.

The hole-dilating balloon 338 is configured to transition between a radially deflated state, shown for example in FIGS. 12A-12C, and a radially inflated state, shown for example in FIG. 12D. The hole-dilating balloon 338 is configured to be positioned inside the pilot puncture 50, and expand the pilot puncture to form a tissue opening, such as a leaflet opening 52, as shown in FIG. 12D and explained in greater detail below.

An enlarged view of a distal portion of the dilation apparatus 320 is illustrated in FIG. 11A, extending out of the outer shaft 310 for example. Cross-sectional views of the distal portion of the dilation apparatus 320 are further shown throughout FIGS. 12A-12E. The balloon catheter 332 can define a balloon catheter lumen 334, through which a guidewire 80, and one or more additional shafts of the dilation apparatus 320, can optionally extend. The balloon catheter 332 can extend from a balloon catheter adaptor 342 that includes a first adaptor port 344a configured to receive a guidewire therethrough and a second adaptor port 344b configured to receive fluid from a fluid source.

As shown in FIG. 11A, when a dilation apparatus 320 is used in combination with a steerable delivery apparatus 302 after complete removal of the tissue perforation apparatus 200, the balloon catheter adaptor 342 can be positioned proximal to the handle 304 of the steerable delivery apparatus 302, while the distal end of the dilation apparatus 320 can be slid over a portion of the guidewire 80 extending proximally from the handle 304, through the rear port 308 and into the handle 304, and further advanced over the guidewire 80 and through the outer catheter 310 towards the target tissue, such as host leaflet 10.

The second adaptor port 344b can be fluidly connectable to a fluid source (not shown) for inflating the hole-dilating balloon 338. The fluid source comprises an inflation fluid. The term “inflation fluid”, as used herein, means a fluid (for example, saline, though other liquids or gas can be used) used for inflating the hole-dilating balloon 338. The inflation fluid source is in fluid communication with the balloon catheter lumen 334, such that fluid from the fluid source can flow through the balloon catheter lumen 334 into hole-dilating balloon 338 to inflate it.

In some examples, an inflatable balloon 338 of apparatus 320, utilized as a hole-dilating balloon, can be different from a typical balloon used for expanding balloon-expandable prosthetic valves or stents, in that while a typical valve-expanding balloon is inflatable to a diameter that can allow expansion of a prosthetic valve to a functional diameter thereof, which can be similar to, or greater than (for example, in the case of valve over-expansion) the diameter of the native annulus in which the valve is deployed, the maximum diameter of a hole-dilating balloon 338 can be significantly smaller, configured to increase the size of a pilot puncture 50 to form a larger leaflet opening 52, optionally without tearing the host leaflet 10 (though in some examples, the host leaflet 10 may be still torn by a hole-dilating balloon 338).

In some examples, such as when dilation of a pilot puncture 50 is desired to form a larger leaflet opening 52, without necessarily tearing the leaflet 10, the maximum diameter to which the hole-dilating balloon 338 can be inflated is equal to or less than 12 mm. In some examples, the maximum diameter to which the hole-dilating balloon 338 can be inflated is equal to or less than 10 mm. Nevertheless, as mentioned above, in some examples a hole-dilating balloon 338 can be configured to tear a host leaflet 10, in which case the maximum diameter to which the hole-dilating balloon 338 can be greater than 12 mm., such as in a range of 20-25 mm.

In some examples, a dilation apparatus 320 can further include a dilator 322 that can be conical or frustoconical in shape, and include a dilator tapering portion 326 terminating at a dilator distal end 324, and a dilator proximal portion 328 that can be coupled to a dilator shaft 340 that extends proximally therefrom. A dilator lumen 330 continuously extends through the dilator shaft 340 and the dilator 322, open ended at the dilator distal end 324. Attachment of the dilator shaft 340 to the dilator proximal portion 328 can be achieved by a variety of methods, such as overmolding, radio-frequency welding, through an adhesive, and/or a combination thereof. In some examples (not illustrated), the dilator shaft 340 can extend through the entire length of the dilator 322, such that a distal end of the dilator shaft 340 is aligned with the dilator distal end 324. In some examples (not illustrated), the dilator shaft 340 is coupled to one or more components, such as collars or other connectors, which are in turn attached to the dilator 322.

In some examples, the hole-dilating balloon 338 is coupled to a distal end portion of the balloon catheter 332 at its proximal end, while the balloon's distal end can be coupled, directly or indirectly, to another component of the apparatus 320, such as the dilator 322 or dilator shaft 340. In the examples illustrated in FIGS. 11A-12E, the hole-dilating balloon 338 is shown to be coupled to the dilator proximal portion 328. The dilator proximal portion 328 can optionally include an outer step configured to accommodate the distal end of the hole-dilating balloon 338, such that the outer surface of the hole-dilating balloon 338 can be flush or otherwise relatively continuous with the outer surface of the dilator 322.

In some examples, such as when the hole-dilating balloon 338 is attached at both ends thereof to the dilator 322 and balloon catheter 332, both the dilator 322 with dilator shaft 340 and the balloon catheter 332 can be configured to move simultaneously in the axial direction, without necessarily being axially movable relative to each other, or while axial movement of one relative to the other is limited. In such examples, axial movement of the balloon catheter 332 can cause the dilator shaft 340 to move therewith, or axial movement of one of the dilator shaft 340 or dilator 322 can cause the balloon catheter 332 to move therewith.

The dilator shaft 340 can extend through the balloon catheter lumen 334, and may be sized such that an annular space is formed within balloon catheter lumen 334 between an inner surface of the balloon catheter 332 and an outer surface of the dilator shaft 340 along the length of balloon catheter 332. This annular space is in fluid communication with one or more inflation openings 336 exposed to an internal cavity of the hole-dilating balloon 338, which can be in fluid communication, via adaptor port 344b of balloon catheter adaptor 342, with a fluid source (for example, a syringe or a pump) that can inject inflation fluid (for example, saline) into the hole-dilating balloon 338, so as to inflate the balloon 338, for example during formation of a leaflet opening 52 as will be described in greater detail below and shown, for example, in FIG. 12D. The pressure of the inflation fluid within hole-dilating balloon 338 may provide the force that allows it to dilate a leaflet opening 52. Further, the balloon catheter lumen 334 may be configured to withdraw fluid from the balloon 338 through the inflation opening(s) 336, to deflate the balloon 338.

In some examples, the handle 202 of a tissue perforation apparatus 200 can include a handle distal portion 294 and a handle proximal portion 296 which are separable from each other. A knob of the handle 202, such as a rotatable knob 204a, can be configured to control a lock and release mechanism configured to keep both handle portions 294, 296 attached to each other during utilization of the tissue perforation apparatus 200 for forming a pilot puncture, as described for example above with respect to FIGS. 8A-10D, and to allow separation of the handle proximal portion 296 from the handle distal portion 294 after formation of the pilot puncture 50 is complete and retrieval of the needle 212 and anchor device 230 is desired.

The handle proximal portion 296 can include the knobs 204b and 204c for controlling rotational and axial movements of the anchor device 230 and the needle 212, wherein proximal portions of the anchor shaft 242 and the needle shaft 222 can be coupled to the handle proximal portion 296, such as to mechanisms of the handle proximal portion 296 controllable by the knobs 204b, 204c. The delivery catheter 206 is attached to the handle distal portion 294, while the anchor device 230 and needle 212 can extend through the handle distal portion 294 to connect with handle proximal portion 296, without being attached to the handle distal portion 294 itself. Thus, when the knob 204a is actuated to separate the handle portions 294 and 296, the anchor device 230 and the needle 212 can be removed, along with the handle proximal portion 296, from the handle distal portion 294.

In some examples, after forming the pilot puncture 50 and rotating the anchor device 230 to release it from engagement with the host leaflet 10, the knob 204a can be actuated to allow separation of the handle proximal portion 296 from the handle distal portion 294. The handle proximal portion 296 can be then proximally pulled, while the handle distal portion 294 remains in position, such that the anchor device 230 and the needle 212 can be retracted through the delivery catheter lumen 208 and out of the handle 304 of steerable delivery apparatus 302, while the delivery catheter 206 can remain in position, extending through the outer catheter 310 towards the host leaflet 10. In such examples, as shown in FIG. 11B, the dilation apparatus 320 can be inserted into the delivery catheter 206 and advanced through the delivery catheter lumen 208, optionally over the guidewire 80, towards the host leaflet 10.

In some cases, a steerable outer catheter 310 of the steerable delivery apparatus 302 can be steered towards the site of treatment so as to generally face the host valvular structure 12. The smaller-sized delivery catheter 206 can be then extended out of the outer catheter 310, and may be more easily oriented towards a specific desired host leaflet 10 in which a pilot puncture needs to be formed. In such cases, insertion of the balloon catheter 332 into the delivery catheter 206 as exemplified in FIG. 11B, can facilitate easier navigation thereof towards the specific host leaflet 10 and its pilot puncture 50, compared to direct insertion of the balloon catheter 332 into an outer shaft 310 from which the delivery catheter is removed, as exemplified in FIG. 11A.

While a dilation apparatus 320 is shown in FIGS. 11A-11B to extend through an outer shaft 310 and over a guidewire 80 that remain in situ after retraction of the anchor device 230 and needle 212, either directly inserted through the handle 304 of apparatus 302 after removal of the delivery catheter 206 as well, as shown in FIG. 11A, or inserted into a delivery catheter 206 that remains in position inside the outer catheter 310, as shown in FIG. 11B, it is to be understood that these configurations are shown by way of illustration and not limitation. In some examples, the guidewire 80 can be retrieved from the patient's body while the outer catheter 310 remains in situ, with the delivery catheter 206 either retrieved as well or remaining inside the outer catheter, prior to insertion of the balloon catheter 332. The same guidewire 80 or a different guidewire can be then optionally reinserted, for example through the first adapter portion 344a of the balloon catheter adaptor 342, over which the balloon catheter 332 can be guided towards the pilot puncture 50 of the host leaflet 10, through the outer catheter 310 and/or delivery catheter 206.

In some examples, both the tissue perforation apparatus 200 and the steerable delivery apparatus 302 can be retrieved from the patient's body, while only the guidewire 80 can remain in situ, optionally extending through the pilot puncture, such that the balloon catheter 332 can be guided over the guidewire 80 towards the host leaflet 10.

FIGS. 12A-12E illustrate some steps in a method for utilizing a dilation apparatus 320 for forming an opening by dilating a previously formed puncture in a target tissue, such as a host leaflet 10. Subsequent to forming the pilot puncture 50 and after retraction of the anchor device 230 and needle 212, the hole-dilating balloon 338, carried over the balloon catheter 332, can be advanced towards the host leaflet 10 according to any of the methods described above. Thus, while the balloon catheter 332 is illustrated in FIGS. 12A-12E to extend through an outer catheter 310, it is to be understood that this is shown by way of illustration and not limitation, and that the balloon catheter can extend similarly through a delivery catheter 206 that can optionally extend through the outer catheter 310, or it can be advanced towards the host leaflet 10 without passing through any of the outer catheter 310 and/or delivery catheter 206.

In some examples, when the dilation apparatus 320 further includes a dilator 322 as also shown in the example illustrated in FIG. 12A, the dilator 322 can be advanced, optionally along with the balloon catheter 332 and hole-dilating balloon 338, towards the host leaflet 10. When included in dilation apparatus 320, the dilator 322 can be inserted into the pilot puncture 50 to expand the pilot puncture 50, as shown in FIG. 12B. As the dilator 322 is inserted into the host leaflet 10, the inherent resiliency of the leaflet 10 may urge the leaflet 10 radially inwardly against the dilator 322. The dilator 322 can have sufficient stiffness to facilitate advancement thereof through the leaflet 10, wherein the gradually tapering shape of the dilator 322 facilitates expanding the pilot puncture 50 to a greater diameter.

In a subsequent step of the method, illustrated in FIG. 12C, the hole-dilating balloon 338 may be inserted within the pilot puncture 50, such as by further advancement of the dilator 322 with dilator shaft 340 and/or balloon catheter 332. With the hole-dilating balloon 338 received within the pilot puncture 50, inflating the hole-dilating balloon 338 to transition it from a radially deflated state (FIG. 12C) to a radially inflated state (FIG. 12D) can expand the pilot puncture 50 to form a leaflet opening 52 that is sized to receive the prosthetic valve 100 in the radially compressed or crimped configuration. After the hole-dilating balloon 338 is inflated to form the leaflet opening 52 as shown in FIG. 12D, the hole-dilating balloon 338 is deflated, as shown in FIG. 12E, optionally allowing for insertion of a guest prosthetic valve inside the leaflet opening 52.

In some examples, inflating the hole-dilating balloon 338 within the host leaflet 10 serves to increase a diameter of the pilot puncture 50 such that the resulting leaflet opening 52 is a hole with an increased diameter relative to the pilot puncture 50. In some examples in which the leaflet opening 52 is a hole, the leaflet opening 52 may be a substantially circular hole. In some examples, the leaflet opening 52 may be non-circular (for example, elliptical or asymmetric). In such examples, the diameter of the leaflet opening 52 may refer to any suitable dimension of the leaflet opening 52, such as a minimum diameter of the leaflet opening 52, a maximum diameter of the leaflet opening 52, and/or an average diameter of the leaflet opening 52.

In some examples, inflating the hole-dilating balloon 338 within the host leaflet 10 may cause the host leaflet 10 to rip and/or tear such that the leaflet opening 52 is not a bounded hole. Stated differently, in such examples, the leaflet opening 52 may be formed by a tear that extends from the pilot puncture 50 fully to the free edge of the host leaflet 10 (the coaptation edge of the leaflet).

While a dilation apparatus 320 that includes a hole-dilating balloon 338 is described above and illustrated for expanding a pilot puncture 50 to form a leaflet opening 52, it is to be understood that other types of expansion member can be used instead of a balloon in any of the methods and/or systems described herein. For example, U.S. Provisional Application No. 63/335,739, which is incorporated herein by reference in its entirety, describes an expandable frame that can be used as an expansion member instead of a valve-expanding balloon.

In some examples, retraction of the hole-dilating balloon 338, after deflation thereof, can be performed while the guidewire 80 may be kept in position, extending through the leaflet opening 52. Subsequent to deflation of the hole-dilating balloon 338 (or recompressing of any other type of an expansion member) inside the leaflet opening 52 and retracting it away from the host leaflet 10, the method can further include steps of positioning a guest prosthetic valve 100 inside the leaflet opening 52. A replacement valve delivery apparatus 350 carrying the guest prosthetic valve 100 can be either part of the system 300, or provided as a separate assembly of apparatus 350 advanced into a leaflet opening 52.

FIG. 12F shows a guest prosthetic valve 100 positioned, in a radially compressed configuration thereof, inside the leaflet opening 52. As shown in FIG. 12F, the guest prosthetic valve 100 can be mounted on a replacement valve delivery apparatus 350 that can be advanced towards the host leaflet 10 over a guidewire, which can be a separate guidewire (not shown), or can be the same guidewire 80.

In some examples, the guest prosthetic valve is a balloon expandable valve, and the replacement valve delivery apparatus 350 comprises a balloon catheter 352 carrying a valve-expanding balloon 354. In contrast to some examples of a hole-dilating balloon 338 described above, such as a hole-dilating balloon 338 configured to form a leaflet opening 52 without tearing the host leaflet, the maximum diameter to which a valve-expanding balloon 354 can be inflated can be, in some examples, greater than 18 mm., greater than 20 mm., greater than 23 mm., greater than 26 mm., and/or greater than 29 mm.

While a replacement valve delivery apparatus 350 equipped with a valve-expanding balloon 354 at a distal end portion of a balloon catheter 352 is illustrated, it is to be understood that this is shown by way of illustration and not limitation, and that a replacement valve delivery apparatus 350 can include other shafts and/or mechanisms, for example when utilized to advance and expand other types of replacement prosthetic valves, such as self-expandable prosthetic valves or mechanically expandable prosthetic valves.

In some examples, the replacement valve delivery apparatus 350 can further include a nosecone 356 positioned distal to the valve-expanding balloon 354 (or other prosthetic-valve expanding mechanism). The nosecone 356 can be conical or frustoconical in shape. The nosecone 356 can be attached to a distal end of a nosecone shaft 358 extending through the balloon catheter 352, wherein the nosecone 356 and the nosecone shaft 358 can collectively define a lumen through which a guidewire can extend. In some examples, when a nosecone 356 is present at a distal end of the replacement valve delivery apparatus 350 as also shown in the example illustrated in FIG. 12F, the nosecone 356 can be advanced towards the host leaflet 10, and may optionally have a maximal diameter that can be somewhat greater than the diameter of the opening 52, such that as the nosecone 356 is inserted into the leaflet opening 52 it can optionally further expand the leaflet opening 52 to a greater diameter.

As shown in FIG. 12F, the guest prosthetic valve 100 is placed in the leaflet opening 52 in its radially compressed configuration, optionally positioned over a deflated valve-expanding balloon 354 in the case of a balloon-expandable prosthetic valve. With the prosthetic valve 100 received within the leaflet opening 52, radially expanding the guest prosthetic valve 100, as shown in FIG. 12G, can serve to increase a size of the leaflet opening 52 and/or to tear the leaflet. As a result, and as discussed above, radially expanding the guest prosthetic valve 100 can serve to modify the host leaflet 10 such that the leaflet does not obstruct a cell opening 112 in a frame 102 of the guest prosthetic valve 100 or at least increases the area of the host valve and the guest valve that is not covered or obstructed by the modified host leaflet to permit access and sufficient perfusion to the adjacent coronary artery. For example, radially expanding the guest prosthetic valve within the leaflet opening 52 can operate to push a portion of the leaflet extending radially exterior of the guest prosthetic valve below an upper edge of an outer skirt of the guest prosthetic valve 100 and/or away from one or more cell openings 112 of the guest prosthetic valve 100.

In some examples, the guest prosthetic valve can be a mechanically-expandable prosthetic valve and radial expansion thereof can be achieved by actuating a mechanical actuator of the guest prosthetic valve to mechanically expand a frame of the guest prosthetic valve. In some examples, the guest prosthetic valve can be a self-expandable prosthetic valve that can be retained during delivery toward the host valvular structure in a capsule or other restraint disposed therearound, and valve expansion can be achieved by removing the capsule or other restraint from the guest prosthetic valve to allow it to radially self-expand within the host valvular structure.

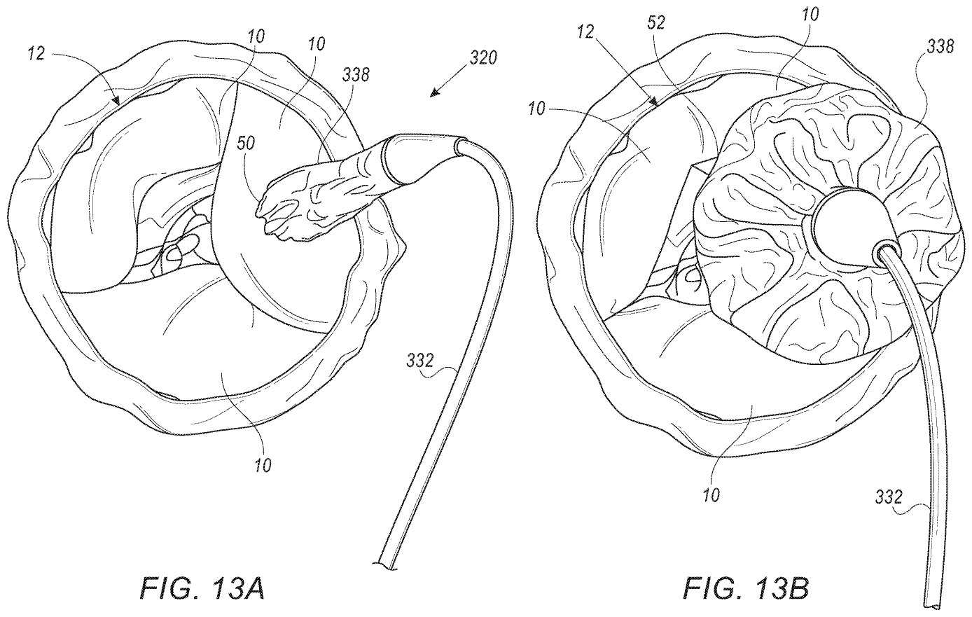

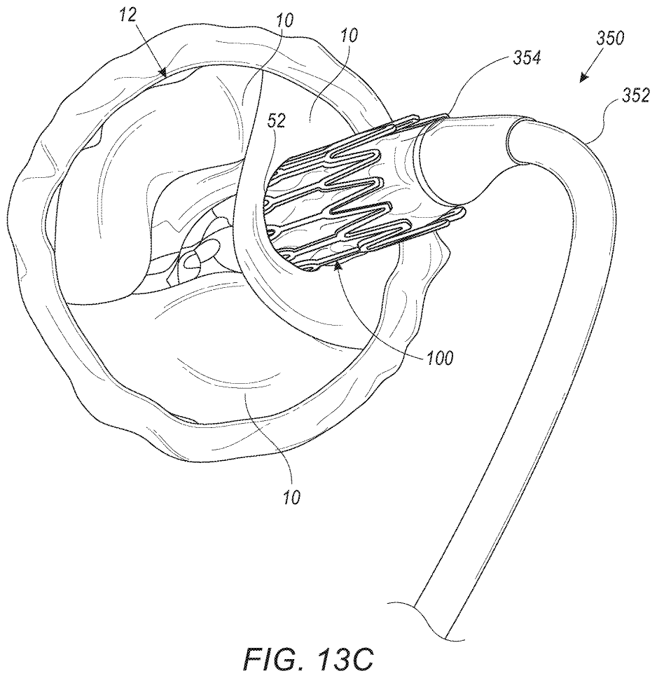

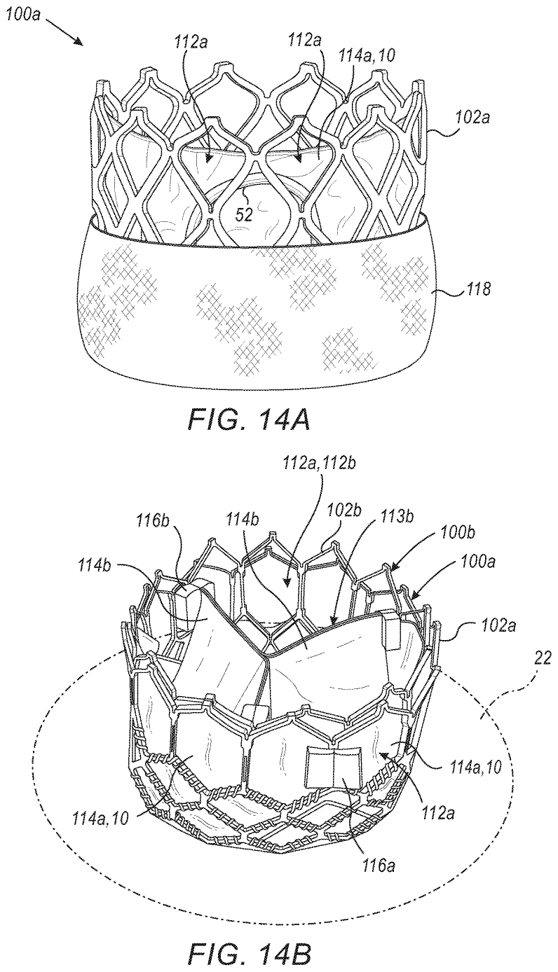

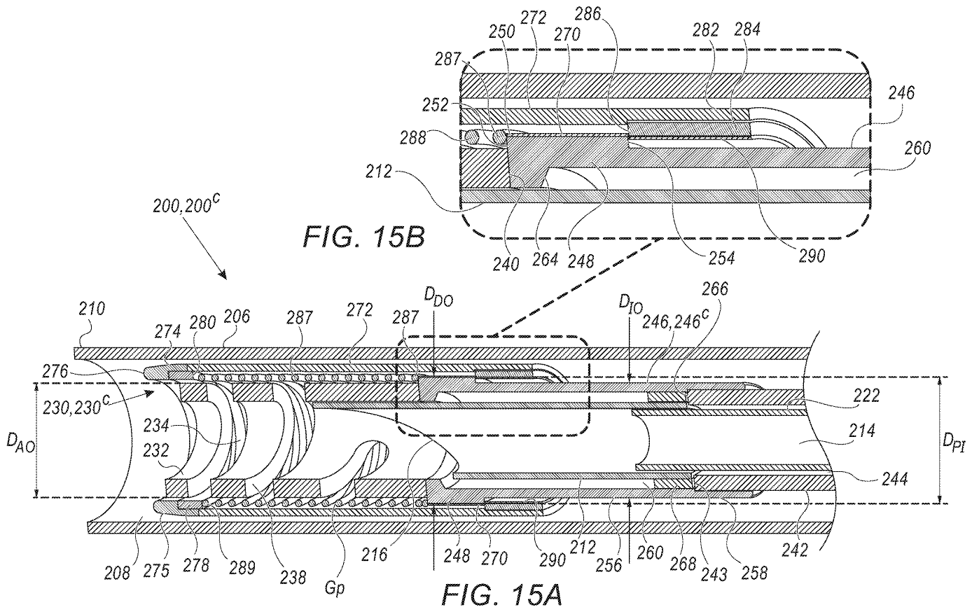

FIGS. 13A-14B illustrate a sequence of events in which a host valvular structure 12 is modified to receive a guest prosthetic valve 100. FIGS. 13A-13B illustrate the hole-dilating balloon 338 utilized to expand the pilot puncture 50 into the leaflet opening 52. In particular, FIG. 13A illustrates the hole-dilating balloon 338 in a deflated state within the pilot puncture 50, corresponding to the state described above with respect to FIG. 12C, while FIG. 13B illustrates the hole-dilating balloon 338 in an inflated state such that the pilot puncture 50 has enlarged into the leaflet opening 52, corresponding to the state described above with respect to FIG. 12D. FIG. 13C illustrates a guest prosthetic valve 100 that can be positioned in the leaflet opening 52 after removal of the hole-dilating balloon 338 therefrom, in a crimped configuration of the prosthetic valve 100, corresponding to the state described above with respect to FIG. 12F, after which the guest prosthetic valve 100 can be expanded, such as by inflating a valve-expanding balloon 354 over which it can be mounted in the case of a balloon-expandable valve, so as to implant the guest prosthetic valve 100 inside the host valvular structure 12.

As mentioned, any system, apparatus and method of the current specification can be utilized for forming a leaflet opening 52 in a host leaflet 10 which can be either a native leaflet 30 or a prosthetic valve leaflet 114 of a previously implanted prosthetic valve, such as prosthetic valve 100a of FIG. 3, such as in the case of ViV procedures. FIG. 14A shows a previously implanted prosthetic valve 100a subsequent to forming the leaflet opening 52. FIG. 14B shows a configuration in which a second prosthetic valve 100b has been expanded within the leaflet opening 52 of a host prosthetic valve 100a. In the example of FIG. 14B, the guest prosthetic valve 100b is the same type of valve as the host prosthetic valve 100a. It is to be understood, however, that ViV procedures may be similarly applied to any other suitable valvular structures, such as different prosthetic valves and/or native heart valves. For example, the guest prosthetic valve 100b need not be the same type of valve as the host prosthetic valve 100a.

In the example of FIG. 14A, when the prosthetic valve leaflets 114a of the previously implanted prosthetic valve 100a are pressed against the frame 102a, the leaflet opening 52 provides a partial access into the frame 102a, but the leaflet opening 52 may not be sufficiently large to completely uncover any of the cell openings 112a of the frame 102a.

As shown in FIG. 14B, however, fully expanding the guest prosthetic valve 100b within the leaflet opening 52 further expands and/or tears the leaflet opening 52 such that several cell openings 112a of the frame 102a of the host prosthetic valve 100a and several cell openings 112b of the frame 102b of the guest prosthetic valve 100b are fully uncovered by the leaflets 114a. In some examples, this may result from the frame 102b of the guest prosthetic valve 100b pushing the leaflet 114a comprising the leaflet opening 52 downwardly (toward the inflow ends of the prosthetic valves 100a, 100b) such that one or more cell openings 112a are unobstructed by the leaflet 114a. In some examples, expanding the frame 102b within the leaflet 114a comprising the leaflet opening 52 may rip and/or tear this leaflet 114a such that the leaflet 114a cannot obstruct one or more cell openings 112a.

FIG. 15A is a cross-sectional view of a distal portion of an exemplary tissue perforation apparatus 200^{c}. Tissue perforation apparatus 200^{c} comprising an anchor device 230^{c} with a connector 246^{c} are exemplary implementations of a tissue perforation apparatus 200 comprising an anchor device 230 with a connector 246, and thus can include any of the features described for apparatus 200 comprising an anchor device 230 and connector 246 throughout the current disclosure, except that the tissue perforation apparatus 200^{c} further comprises an anchoring verification mechanism, which includes a plunger 272 having a plunger marker 290, which is axially movable with respect to an anchor marker 270 of connector 246^{c}. The position of the plunger marker 290 relative to the anchor marker 270 can be indicative of successful or failed anchoring of the helical anchor 232 against the target tissue, such as a host leaflet 10. FIG. 15B shows an enlarged view of a region of FIG. 15A including the connector distal end portion 248 and a proximal end portion 282 of the plunger 272.

The plunger 272 can be part of the anchor device 230^{c}, and can be disposed around at least part of the helical anchor 232 and at least part of the connector 246^{c}. The plunger 272 extends between a distal end portion 274 thereof and a proximal end portion 282 thereof. In some examples, the helical anchor 232 defines an outer diameter D_{AO}, and the plunger 272 has an inner diameter that is greater than the anchor's outer diameter D_{AO.} In some examples, at least a portion of the plunger extending proximally from the plunger distal end portion 274 can have an inner diameter is greater than the anchor's outer diameter D_{AO.} The helical anchor 232 and the plunger 272 can together define a radial plunger gap G_{P} between an inner surface of the plunger 272 and an outer surface of the anchor 232.

The connector distal end portion 248 can have an outer diameter D_{DO} that is greater than the outer diameter D_{AO} of the anchor 232, so as to define a connector distal outer step 252 between a distal end 250 of the connector 246 and the helical anchor proximal end 240.

The plunger distal end portion 274 can protrude radially inwards, so as to form a plunger distal step 280 at the distal end of the plunger gap G_{P}. In some examples, the plunger 272 can include a distal ring 278 at the plunger distal end portion 274, configured to protrude radially inwards so as to form the plunger distal step 280. In some examples, the distal ring 278 can be provided as a separate component that can be affixed to the plunger distal end portion 274, such as by being glued, welded, soldered, or otherwise attached to an inner surface of the plunger distal end portion 274. In some examples, the inwardly-oriented radial protrusion of the plunger distal end portion 274 is an integrally formed part of the plunger distal end portion 274, instead of being a separate component affixed to the plunger distal end portion 274.

In some examples, the plunger distal end portion 274 can define a tapering outer surface 275 terminating at a distal end 276 of the plunger 272. In some examples, the plunger distal end 276 is an atraumatic distal end, such as by being rounded and/or being curved radially inwards.

The anchor device 230^{c} further comprises a spring 288 disposed inside the plunger gap G_{P} between the plunger 272 and the helical anchor 232, axially extending between the connector distal outer step 252 and the plunger distal step 280. The spring 288 has a spring proximal end 287 that can be attached to, or in contact with and pressed against, the connector distal outer step 252, and a spring distal end 289 that can be attached to, or in contact with and pressed against, the plunger distal step 280. As shown in FIG. 15A, the spring 288 is configured, in a free state thereof, to bias the plunger 272 distally, relative to the connector 246^{c}.

As further shown in FIG. 15A, the connector intermediate portion 256 defines an outer diameter D_{IO} which is smaller than the outer diameter D_{DO} of the connector distal end portion 248, so as to define an outer proximally-facing step 254 between the connector intermediate portion 256 and the connector distal end portion 248.

The connector proximal end portion 258 can be defined as the portion of the connector 246^{c} attached to the anchor shaft 242, such that the chamber proximal step 266, optionally defined by the anchor shaft distal end 243, can be at the transition from the connector proximal end portion 258 to the connector intermediate portion 256. In some examples, the outer diameter along the connector proximal end portion 258 can be equal to the outer diameter D_{IO} of the connector intermediate portion 256.

The plunger proximal end portion 282 can protrude radially inwards, so as to form an inner distally-facing step 286 at the distal end of the plunger proximal end portion 282. In some examples, the plunger 272 can include a proximal ring 284 at the plunger proximal end portion 282, configured to protrude radially inwards so as to form the inner distally-facing step 286. In some examples, the proximal ring 284 can be provided as a separate component that can be affixed to the plunger proximal end portion 282, such as by being glued, welded, soldered, or otherwise attached to an inner surface of the plunger proximal end portion 282. In some examples, the inwardly-oriented radial protrusion of the plunger proximal end portion 282 is an integrally formed part of the plunger proximal end portion 282, instead of being a separate component affixed to the plunger proximal end portion 282.

The plunger proximal end portion 282 can protrude radially inwards to an inner diameter that is smaller than the outer diameter D_{DO} of the connector distal end portion 248, such that when the spring 288 extends to distally bias the plunger 272 in its free state, the inner distally-facing step 286 of the plunger 272 abuts outer proximally-facing step 254 of connector 246^{c}, thereby limiting the extent to which the plunger 272 can be distally biased by the spring 288.

It is to be understood that any reference herein to the spring 288 being in a free state thereof, refers to the spring 288 biasing the plunger 272 to its distal-most position, which can be also referred to as a distally-biased position of the plunger 272, as shown in FIG. 15A, without the spring 288 being forced to compress due to the distal end 276 of the plunger 272 being pressed against any other structure or component.

The plunger distal end 276 can be, at or distal to, the anchor tip 236, in the distally-biased position of the plunger 272. While the plunger 272 can extend past the helical anchor 232 such that the plunger distal end 276 is positioned distal to the anchor tip 236 in the distally-biased position illustrated for example in FIG. 15A, it is to be understood that in some examples, the plunger distal end 276 can be aligned with the anchor tip 236 in the distally-biased position.

The connector 246^{c} can further include a radiopaque anchor marker 270, for example positioned at the connector distal end portion 248. In some examples, the radiopaque anchor marker 270 can span at least part of, or the entirety of, the circumference of the connector distal end portion 248. In some examples, the radiopaque anchor marker 270 can extend distally from the axial position of the outer proximally-facing step 254. In some examples, the radiopaque anchor marker 270 can extend from the outer proximally-facing step 254 to the connector distal end 250.

The plunger 272 can further include a radiopaque plunger marker 290, for example positioned at the plunger proximal end portion 282. In some examples, the radiopaque plunger marker 290 can span at least part of, or the entirety of, the circumference of the plunger proximal end portion 282. In some examples, the radiopaque plunger marker 290can extend proximally from the axial position of the inner distally-facing step 286.

Each of the radiopaque markers 270, 290 comprises a radiopaque material, understood to be capable of producing a relatively bright image on a fluoroscopy screen or another imaging technique, during tissue puncturing or opening formation procedure by tissue perforation apparatus 200^{c}. Any of the radiopaque markers 270, 290 can comprise any material or combination of materials that are radiopaque or increase the radiopacity of at least a portion of the connector 246 and the plunger 272 comprising the corresponding markers 270 and 290. Radiopaque materials can include, but are not limited to, gold, platinum, tantalum, tungsten alloy, platinum iridium alloy, palladium, barium sulfate, bismuth, and/or any other material which is opaque to fluoroscopy, X-rays, or similar radiation or any combination thereof.

In some examples, any of the radiopaque markers 270, 290 can have a higher radiopacity than one or more other components of the tissue perforation apparatus 200^{c}, such as any other portion of the plunger 272, and/or any other portion of the connector 246. In some examples, any of the radiopaque markers 270, 290 can have a higher radiopacity than the needle 212. In some examples, any of the radiopaque markers 270, 290 can have a higher radiopacity than the delivery catheter 206.

In some examples, any of the radiopaque anchor marker 270 and/or 290 can be formed by means of radiopaque inks and adhesives, and applied on the corresponding anchor device components in a number of ways, such as screen printing, high speed roller printing, coating, dipping, etc. In some examples, any of the radiopaque anchor marker 270 and/or 290 can be separately formed components, such as annular rings or bands that are mounted on or inside the corresponding anchor device components.

In some examples, the plunger marker 290 is proximal to the anchor marker 270, and the axial distance between the markers 290 and 270 can vary due to relative axial movement between the anchor 232 and the plunger 272 relative to each other. In some examples, a radiopaque anchor marker 270 can be located at the connector distal end portion 248. In some examples, a radiopaque plunger marker 290 can be located at the plunger proximal end portion 282.

In some examples, a radiopaque anchor marker 270 can be embedded in, attached to, or coated over a surface of, the connector distal end portion 248. In some examples, the radiopaque anchor marker 270 can be disposed along an outer surface of the connector distal end portion 248. In some examples, the outer diameter D_{DO} of the connector distal end portion 248 includes a thickness of the radiopaque anchor marker 270, and can be defined, in some cases, as the outer diameter of the radiopaque anchor marker 270.

In some examples, a radiopaque plunger marker 290 can be embedded in, attached to, or coated around a surface of, the plunger proximal end portion 282. In some examples, the radiopaque plunger marker 290 can be disposed along an inner surface of the plunger proximal end portion 282. In some examples, a radiopaque plunger marker 290 can be embedded in, attached to, or coated around a surface of, a proximal ring 284 attached of the plunger proximal end portion 282. In some examples, the radiopaque plunger marker 290 can be disposed along an inner surface of the proximal ring 284.

In the distally-biased position of the plunger 272, as shown in FIG. 15A, the plunger proximal end portion 282 can abut connector distal end portion 248, such that the plunger marker 290 and anchor marker 270 are in close proximity to each other. In some examples, the plunger marker 290 can proximally extend from the inner distally-facing step 286 and the anchor marker 270 can distally extend from the outer proximally-facing step 254, such that markers 270, 290 are axially continuous with each other. This can allow the markers 270, 290 to together form an elongated radiopaque imagery, having an overall axial length corresponding to the combined lengths of the markers 290 and 270 under fluoroscopy, X-rays, or similar radiation-based imaging modality.

FIG. 16A shows a cross-sectional view of a distal portion of the tissue perforation apparatus 200^{c} of FIGS. 15A-15B approaching a target tissue, such as a host leaflet 10. This state, wherein the tissue perforation apparatus 200^{c} does not yet contact the host leaflet 10, can be representative of the tissue perforation apparatus 230^{c} during delivery towards the host leaflet 10, similar to that described above with respect to FIG. 8A for example. In this state, the helical anchor 232 can be retained inside the delivery catheter 206, and the needle tip 220 can be positioned proximal to the anchor tip 236.

FIG. 16B shows the distal portion of the tissue perforation apparatus 200^{c} in contact with the host leaflet 10, wherein the helical anchor 232 did not penetrate yet into the tissue material. For example, the delivery catheter distal end 210 can be slightly pressed against the host leaflet 10 as described above. As long as the anchor 232 does not extend through the host leaflet 10, as shown in FIG. 6A or 6B, the plunger is in its distally-biased position, such that the radiopaque markers 290 and 270 are in close proximity to each other, and may be optionally visible, under fluoroscopy, X-rays, or similar radiation-based imaging modality, as a relatively continuous elongated radiopaque marking imagery.

As shown in FIG. 16A, the spring 288 can have a first length L1 between the spring proximal end 287 and the spring distal end 289, or between the connector distal outer step 252 and the plunger distal step 280, in the distally-biased position of the plunger 272.

When the helical anchor 232 is rotated and successfully penetrates through the host leaflet 10, as shown in FIG. 6C, the connector 246, which is affixed to the anchor 232, is axially translated in the distal direction along with the anchor. However, the plunger 272 surrounding the anchor 232 cannot move distally along with the anchor 232 and the connector 246, since the tissue material of the host leaflet 10 surrounding the anchor 232 prevents the plunger's distal end 276 from passing therethrough. Thus, as the connector 246 move distally during anchor 232 penetration, the spring 288 is forced to compress between the distal outer step 252 and the surface of the host leaflet 10, to a second length L2 which is shorter than the first length L1. Thus, the first length L1 can be defined as a pre-penetration length, and the second length L2 can be defined as a post-penetration length.

A spring force constant of the spring 288 can be selected to bias the plunger 272 distally to the position shown in FIGS. 6A-6B, for example, in its free state, yet allow the spring 288 to compress when the plunger distal end 276 is pushed against a leaflet 10 during anchoring of the helical anchor 232 to the leaflet 10. It is to be understood that a spring 288 can be replaced by any equivalent biasing member known in the art.

As the plunger 272 cannot move distally relative to the leaflet 10, while the connector 246 moves in the distal direction along with the anchor 232, the anchor marker 270 at the connector's distal end portion 248 moves distally away from the plunger marker 290, resulting in an axial separation between both markers 270, 290 by a separation distance S_{M}, which can be equal to the difference between the first length L1 and the second length L2 of the spring 288 (i.e., S_{M}=L1−L2).

When viewed under fluoroscopy, X-ray, or similar radiation-based imaging modality, separation between both markers 290 and 270, such as along a separation distance S_{M}, can be indicative of successful penetration of the anchor 232 into the host leaflet 10. In contrast, if, following rotation of the anchor 232 to secure it to the leaflet 10, the markers 290 and 270 remain in close proximity to each other, this may be indicative of failed penetration attempt, wherein the anchor 232 remain substantially proximal to the leaflet 10, equivalent to the states illustrated in any of FIG. 6A or 6B.

Thus, utilization of a tissue perforation apparatus 200^{c} having the above-described anchor securement verification mechanism, can be used as an alternative to the method described above with respect to FIGS. 9A-9B for example. If successful engagement of the anchor 232 with the host leaflet 10 is detected, as described above with respect to FIG. 16C, leaflet opening 52 formation and optional guest prosthetic valve 100 implantation can be followed according to any exemplary method steps described above with respect to FIGS. 10A-14B, mutatis mutandis.

While the anchor marker 270 is illustrated and described herein to be located at the connector distal end portion 248, it is to be understood that in some examples, the anchor marker 270 can be located at any portion of the connector 246 or the helical anchor 232 to which it is affixed, such that axial movement of the helical anchor 232 or any component affixed thereto, such as connector 246, will cause the anchor marker 270 to move therewith. For example, an anchor marker can be located at the helical anchor proximal end 240 instead of, or in addition to, the connector distal end portion 248.

While the plunger marker 290 is illustrated and described herein to proximal to the anchor marker 270 prior to anchoring the helical anchor 232 to the leaflet 10, it is to be understood that the plunger marker 290 can be located at any portion of the plunger 272. In some examples, the plunger marker 290 can be positioned distal to the anchor market 270 prior to anchoring the helical anchor 232 to the leaflet 10, in which case, upon anchoring of the helical anchor 232 to the target tissue, such as leaflet 10, the distance between the markers 270 and 290 can decrease. In some examples, the markers 270 and 290 can have a shorter separation distance S_{M} when the helical anchor 232 is anchored to the leaflet 10. In some examples, the markers 270 and 290 can overlap each other when the helical anchor 232 is anchored to the leaflet 10.

Thus, determination that the distance between the markers 270 and 290 has changed after advancing the helical anchor 232 to secure the anchor device 230 to the target tissue, can be indicative of successful anchoring of the helical anchor 232, wherein the change in distance depends on the relative position between the markers 270, 290 prior to advancing the helical anchor 232 to secure the anchor device 230, and can include either increasing or decreasing the distance, optionally beyond a threshold distance that can be indicative of successfully penetration and anchoring.

While a tissue perforation apparatus 200 is described herein in as part of a tissue modification system 300 that includes an a steerable delivery apparatus 302 equipped with an outer catheter 310 through which a distal portion of the tissue perforation apparatus 200 can be advanced, it is to be understood that any exemplary tissue perforation apparatus 200 disclosed herein can be used in isolation, and without a separate steerable delivery apparatus 302.

While a tissue perforation apparatus 200 is described herein as part of a tissue modification system 300 that includes a dilation apparatus 320 that includes an expansion member, such as a hole-dilating balloon 338, used to form a tissue opening (e.g., leaflet opening 52) after retraction of the anchor device 230 and needle 212 from a pilot puncture 50 formed thereby, it is to be understood that any exemplary tissue perforation apparatus 200 disclosed herein can be used in isolation, and without a separate dilation apparatus 320. In some examples, any exemplary tissue perforation apparatus 200 disclosed herein can further include an expansion member, such as a hole-dilating balloon 338 mounted on a balloon catheter 332, as part of the tissue perforation apparatus 200 itself.

While a tissue perforation apparatus 200 is described above for use in a method for forming a leaflet opening prior to implantation a guest prosthetic valve 100 inside a host valvular structure 12, it is to be understood that any exemplary tissue perforation apparatus 200 disclosed herein can be used to form a puncture or opening in any target tissue, including, but not limited to, a leaflet, in any other procedure that may not require utilization of an expansion member, such as a hole-dilating balloon 338, to further expand the opening, and may not involve procedural steps of guest prosthetic valve implantation.

Any of the systems, devices, apparatus, etc. disclosed herein can be sterilized (for example, with heat, radiation, and/or chemicals, etc.) to ensure they are safe for use with patients, and any of the methods herein can include sterilization of the associated assembly, device, apparatus, etc. as one of the steps of the method. Examples of radiation for use in sterilization include, without limitation, gamma radiation and ultra-violet radiation. Examples of chemicals for use in sterilization include, without limitation, ethylene oxide and hydrogen peroxide.

### Some Examples of the Disclosed Technology

Some examples of above-described technology are enumerated below. It should be noted that one feature of an example in isolation or more than one feature of the example taken in combination and, optionally, in combination with one or more features of one or more examples below are examples also falling within the disclosure of this application.

Example 1. A tissue perforation apparatus comprising: an anchor device comprising: a helical anchor defining an anchor channel and terminating with an anchor tip; a plunger extending between a plunger distal end portion and a plunger proximal end portion, wherein the plunger is disposed around at least a portion of the helical anchor, and wherein the plunger and the helical anchor are axially movable with respect to each other; a radiopaque anchor marker configured to move along with the helical anchor; and a radiopaque plunger marker configured to move along with the plunger.

Example 2. The apparatus of any example herein, particularly of example 1, wherein the helical anchor is rotatable around a central longitudinal axis of the anchor device.

Example 3. The apparatus of any example herein, particularly of example 1 or 2, wherein the helical anchor is configured to move distally relative to the plunger when the helical anchor is anchored to a target tissue.

4.Example 4. The apparatus of any example herein, particularly of any one of examples 1 to 3, wherein an axial distance between the radiopaque anchor marker and the radiopaque plunger marker is configured to change when the helical anchor is anchored to a target tissue.

Example 5. The apparatus of any example herein, particularly of example 4, wherein the axial distance between the radiopaque anchor marker and the radiopaque plunger marker is configured to increase when the helical anchor is anchored to a target tissue.

Example 6. The apparatus of any example herein, particularly of example 4, wherein the axial distance between the radiopaque anchor marker and the radiopaque plunger marker is configured to decrease when the helical anchor is anchored to a target tissue.

Example 7. The apparatus of any example herein, particularly of example 3 or 4, wherein the target tissue is a leaflet.

Example 8. The apparatus of any example herein, particularly of example 7, wherein the leaflet is a native leaflet of a native heart valve.

Example 9. The apparatus of any example herein, particularly of example 7, wherein is a prosthetic leaflet of a previously implanted prosthetic valve.

Example 10. The apparatus of any example herein, particularly of any one of examples 1 to 9, further comprising a biasing member configured to bias the plunger to a distally-biased position relative to the helical anchor.

Example 11. The apparatus of any example herein, particularly of example 10, wherein the biasing member is a spring.

Example 12. The apparatus of any example herein, particularly of example 10 or 11, wherein the anchor device further comprises an anchor shaft proximal to the helical anchor, the anchor shaft defining an anchor lumen which is in fluid communication with the anchor channel.

Example 13. The apparatus of any example herein, particularly of example 12, wherein the anchor shaft is a flexible torque shaft configured to rotate around a central axis thereof, such that when the anchor shaft is rotated, the helical anchor is configured to rotate therewith.

Example 14. The apparatus of any example herein, particularly of example 12 or 13, wherein the anchor shaft comprises a helical hollow strand tube.

Example 15. The apparatus of any example herein, particularly of any one of examples 12 to 14, wherein the anchor device further comprises a connector disposed between the helical anchor and the anchor shaft.

Example 16. The apparatus of any example herein, particularly of example 15, wherein the connector comprises a connector distal end portion affixed to the helical anchor, a connector proximal end portion affixed to the anchor shaft, and a connector intermediate portion extending between the connector distal end portion and the connector proximal end portion.

Example 17. The apparatus of any example herein, particularly of example 16, wherein an outer diameter of the connector distal end portion is greater than an outer diameter of the helical anchor.

Example 18. The apparatus of any example herein, particularly of example 16 or 17, wherein a distal end of the connector defines a connector distal outer step at a transition between the connector distal end portion and the helical anchor.

Example 19. The apparatus of any example herein, particularly of example 18, wherein the plunger defines a plunger distal step distal to the connector distal outer step.

Example 20. The apparatus of any example herein, particularly of example 19, wherein the plunger distal step is formed by an inner protrusion of the plunger distal end portion.

Example 21. The apparatus of any example herein, particularly of example 19 or 20, wherein the plunger distal step is formed by a distal ring attached to the plunger distal end portion.

Example 22. The apparatus of any example herein, particularly of any one of examples 19 to 21, wherein the biasing member is disposed between the plunger distal step and the connector distal outer step.

Example 23. The apparatus of any example herein, particularly of any one of examples 16 to 22, wherein the outer diameter of the connector distal end portion is greater than an outer diameter of the connector intermediate portion.

Example 24. The apparatus of any example herein, particularly of any one of examples 16 to 23, wherein the connector defines an outer proximally-facing step at a transition between the connector distal end portion and the connector intermediate portion.

Example 25. The apparatus of any example herein, particularly of example 24, wherein the plunger defines an inner distally-facing step proximal to the outer proximally-facing step of the connector.

Example 26. The apparatus of any example herein, particularly of example 25, wherein the inner distally-facing step is formed by an inner protrusion of the plunger proximal end portion.

Example 27. The apparatus of any example herein, particularly of example 25 or 26, wherein the inner distally-facing step is formed by a proximal ring attached to the plunger proximal end portion.

Example 28. The apparatus of any example herein, particularly of any one of examples 25 to 27, wherein the inner distally-facing step of the plunger is configured to abut the outer proximally-facing step of the connector in the distally-biased position of the plunger.

Example 29. The apparatus of any example herein, particularly of any one of examples 16 to 28, wherein the radiopaque anchor marker is located at the connector distal end portion.

Example 30. The apparatus of any example herein, particularly of any one of examples 1 to 29, wherein the radiopaque plunger marker is located at the plunger proximal end portion.

Example 31. The apparatus of any example herein, particularly of any one of examples 1 to 30, wherein the biasing member is disposed inside a plunger gap radially defined between the plunger and the helical anchor.

Example 32. The apparatus of any example herein, particularly of any one of examples 1 to 31, wherein the radiopaque anchor marker and the radiopaque plunger marker are axially continuous with each other in the distally-biased position of the plunger.

Example 33. The apparatus of any example herein, particularly of any one of examples 1 to 32, wherein the plunger distal end portion defines a plunger distal end which is aligned with, or distal to, the anchor tip, in the distally-biased position of the plunger.

Example 34. The apparatus of any example herein, particularly of example 33, wherein the plunger distal end is configured to move to a position which is proximal to the anchor tip.

Example 35. The apparatus of any example herein, particularly of example 33 or 34, wherein the plunger distal end is an atraumatic distal end.

Example 36. The apparatus of any example herein, particularly of any one of examples 1 to 35, wherein the plunger distal end portion comprises a tapering outer surface.

Example 37. The apparatus of any example herein, particularly of any one of examples 16 to 29, wherein the connector defines a chamber terminating at a chamber distal step.

Example 38. The apparatus of any example herein, particularly of any one of examples 1 to 37, further comprising a needle axially movable relative to the anchor device.

Example 39. The apparatus of any example herein, particularly of example 38, wherein the needle comprises a needle head terminating at a needle tip, wherein the needle head is configured to extend through the anchor channel.

Example 40. The apparatus of any example herein, particularly of example 39, wherein the needle further comprises a needle shaft proximally extending from the needle head.

Example 41. The apparatus of any example herein, particularly of example 40, wherein the needle shaft comprises a plurality of circumferential slits.

Example 42. The apparatus of any example herein, particularly of example 41, wherein the needle shaft comprises a needle shaft distal portion comprising the plurality of circumferential slits.

Example 43. The apparatus of any example herein, particularly of example 42, wherein the needle shaft further comprises a needle shaft proximal portion extending proximally from the needle shaft distal portion.

Example 44. The apparatus of any example herein, particularly of example 43, wherein the needle shaft proximal portion is devoid of circumferential slits.

Example 45. The apparatus of any example herein, particularly of example 43 or 44, wherein the proximal portion of the needle shaft comprises a polymeric material.

Example 46. The apparatus of any example herein, particularly of any one of examples 39 to 45, wherein the needle head defines an angled surface terminating at the needle tip.

Example 45. The apparatus of any example herein, particularly of any one of examples 39 to 46, wherein the needle tip is proximal to the anchor tip in the distally-biased position of the plunger.

Example 48. The apparatus of any example herein, particularly of any one of examples 39 to 46, wherein the needle tip is proximal to the helical anchor in the distally-biased position of the plunger.

Example 49. The apparatus of any example herein, particularly of any one of examples 1 to 48, further comprising a delivery catheter, wherein the anchor device extends through a lumen of the delivery catheter.

Example 50. The apparatus of any example herein, particularly of example 49, wherein the delivery catheter terminates at an atraumatic delivery catheter distal end.

Example 51. The apparatus of any example herein, particularly of example 49 or 50, wherein the helical anchor is axially movable relative to the delivery catheter.

Example 52. A method comprising: advancing a tissue perforation apparatus to a target tissue over a guidewire, wherein the tissue perforation apparatus comprises an anchor device and a needle, wherein the anchor device comprises a radiopaque anchor marker configured to move along with helical anchor of the anchor device, and a radiopaque plunger marker configured to move along with a plunger of the anchor device; advancing the helical anchor towards a target tissue to anchor the anchor device to the target tissue; determining that a distance between the radiopaque anchor marker and the radiopaque plunger marker has changed; and upon determining that the distance between the radiopaque anchor marker and the radiopaque plunger marker has changed, advancing the needle to form a pilot opening at the target tissue.

Example 53. The method of any example herein, particularly of example 52, wherein the determining that the distance has changed comprises determining that the distance between the radiopaque anchor marker and the radiopaque plunger marker is greater after the advancing the helical anchor than before the advancing the helical anchor.

Example 54. The method of any example herein, particularly of example 52, wherein the determining that distance has changed comprises determining that the distance between the radiopaque anchor marker and the radiopaque plunger marker is smaller after the advancing the helical anchor than before the advancing the helical anchor.

Example 55. The method of any example herein, particularly of example 53, wherein the radiopaque anchor marker and the radiopaque plunger marker are adjacent each other prior to the advancing the helical anchor.

Example 56. The method of any example herein, particularly of example 53 or 55, wherein the radiopaque anchor marker and the radiopaque plunger marker form a continuous radiopaque image under fluoroscopy, prior to the advancing the helical anchor.

Example 57. The method of any example herein, particularly of example 56, wherein the determining that the distance has changed comprises determining that the radiopaque anchor marker and the radiopaque plunger marker are separated from each other.

Example 58. The method of any example herein, particularly of example 54, wherein the radiopaque anchor marker and the radiopaque plunger marker are distanced from each other prior to the advancing the helical anchor.

Example 59. The method of any example herein, particularly of example 58, wherein the determining that distance has changed comprises determining that the radiopaque anchor marker and the radiopaque plunger marker at least partially overlap each other.

Example 60. The method of any example herein, particularly of any one of examples 52 to 59, wherein, during the advancing the tissue perforation apparatus, the plunger extends around the helical anchor.

Example 61. The method of any example herein, particularly of any one of examples 52 to 60, further comprising, before the advancing the helical anchor, maintaining the plunger in a distally-biased position relative to the helical anchor.

Example 62. The method of any example herein, particularly of example 61, wherein the anchor device further comprises a biasing member configured to maintain the plunger in the distally-biased position before the anchoring the helical anchor.

Example 63. The method of any example herein, particularly of example 62, wherein the anchoring the helical anchor comprises compressing the biasing member.

Example 64. The method of any example herein, particularly of any one of examples 61 to 63, wherein the biasing member is a spring.

Example 65. The method of any example herein, particularly of any one of examples 61 to 64, wherein a distal end of the plunger is axially aligned with, or distal to, an anchor tip of the helical anchor, in the distally-biased position of the plunger.

Example 66. The method of any example herein, particularly of any one of examples 52 to 65, wherein the anchor device further comprises an anchor shaft proximal to the helical anchor, the anchor shaft defining an anchor lumen which is in fluid communication with an anchor channel of the helical anchor.

Example 67. The method of any example herein, particularly of example 66, wherein the advancing the helical anchor to anchor the anchor device comprises rotating the anchor device in a first rotational direction.

Example 68. The method of any example herein, particularly of example 66, wherein the rotating the anchor device comprises rotating the anchor shaft, and wherein the anchor shaft is a torque shaft configured to transmit rotation movement thereof to the helical anchor.

Example 69. The method of any example herein, particularly of example 66 to 68, wherein the anchor device further comprises a connector disposed between the helical anchor and the anchor shaft.

Example 70. The method of any example herein, particularly of example 69, wherein the connector is affixed to the helical anchor.

Example 71. The method of any example herein, particularly of example 69 or 70, wherein the connector comprises the radiopaque anchor marker.

Example 72. The method of any example herein, particularly of any one of examples 69 to 71, wherein the radiopaque anchor marker is located at a connector distal end portion of the connector.

Example 73. The method of any example herein, particularly of example 72, wherein the radiopaque plunger marker is located at a plunger proximal end portion of the plunger.

Example 74. The method of any example herein, particularly of example 73, wherein the plunger proximal end portion is proximal to the connector distal end portion.

Example 75. The method of any example herein, particularly of example 73 or 74, wherein the plunger proximal end portion is configured to abut the connector distal end portion before the anchoring the helical anchor.

Example 76. The method of any example herein, particularly of any one of examples 73 to 75, wherein the anchoring the helical anchor comprises axially distancing between the plunger proximal end portion and the connector distal end portion.

Example 77. The method of any example herein, particularly of any one of examples 52 to 76, wherein the advancing the tissue perforation apparatus comprises maintaining the anchor device inside a lumen of a delivery catheter of the tissue perforation apparatus.

Example 78. The method of any example herein, particularly of example 77, wherein the advancing the tissue perforation apparatus comprises maintaining the helical anchor proximal to a delivery catheter distal end of the delivery catheter.

Example 79. The method of any example herein, particularly of example 77 or 78, further comprising, prior to the advancing the helical anchor, bringing the delivery catheter distal end to contact with the target tissue.

Example 80. The method of any example herein, particularly of any one of examples 52 to 79, wherein the needle comprises a needle head terminating at a needle tip.

Example 81. The method of any example herein, particularly of example 80, wherein the advancing the tissue perforation apparatus comprises maintaining the needle tip proximal to an anchor tip of the helical anchor.

Example 82. The method of any example herein, particularly of example 80 or 81, further comprising, after the determining, forming a pilot puncture in the target tissue by distally advancing the needle relative to the anchor device.

Example 83. The method of any example herein, particularly of example 82, wherein the forming the pilot puncture further comprises penetrating through the target tissue with the needle head.

Example 84. The method of any example herein, particularly of example 82 or 83, further comprising, after the forming the pilot puncture, retracting the tissue perforation apparatus from the target tissue.

Example 85. The method of any example herein, particularly of example 84, wherein the retracting the tissue perforation apparatus comprises proximally pulling the needle away from the target tissue.

Example 86. The method of any example herein, particularly of example 84 or 85, wherein the retracting the tissue perforation apparatus releasing the helical anchor from the target tissue.

Example 87. The method of any example herein, particularly of example 86, wherein the releasing the helical anchor comprises rotating the anchor device in a second rotational direction.

Example 88. The method of any example herein, particularly of any one of examples 84 to 87, further comprising, prior to the advancing the tissue perforation apparatus, advancing an outer catheter of a steerable delivery apparatus to the target tissue.

Example 89. The method of any example herein, particularly of example 88, wherein the advancing the tissue perforation apparatus comprises advancing the tissue perforation apparatus through the outer catheter.

Example 90. The method of any example herein, particularly of example 88 or 89, wherein the retracting the tissue perforation apparatus comprises retrieving the anchor device and the needle, while maintaining the outer catheter in position.

Example 91. The method of any example herein, particularly of example 90, further comprising, after the retrieving the anchor device and the needle, advancing a dilation apparatus comprising an expansion member to the target tissue.

Example 92. The method of any example herein, particularly of example 91, wherein the advancing the dilation apparatus comprises advancing the expansion member through the outer catheter.

Example 93. The method of any example herein, particularly of example 91 or 92, further comprising positioning the expansion member inside the pilot puncture, in a compacted state of the expansion member.

Example 94. The method of any example herein, particularly of example 93, further comprising, prior to the positioning the expansion member inside the pilot puncture, passing a dilator of the dilation apparatus through the pilot puncture, thereby further expanding the pilot puncture.

Example 95. The method of any example herein, particularly of example 94, wherein the dilator comprises a dilator tapering portion terminating at a dilator distal end.

Example 96. The method of any example herein, particularly of any one of examples 93 to 95, further comprising forming a tissue opening inside the target tissue by expanding the expansion member.

Example 97. The method of any example herein, particularly of example 96, further comprising, after the forming the tissue opening, compressing the expansion member.

Example 98. The method of any example herein, particularly of example 97, further comprising, after the compressing the expansion member, retrieving the dilation apparatus.

Example 99. The method of any example herein, particularly of example 98, wherein the expansion member is a hole-dilating balloon mounted on a balloon catheter of the dilation apparatus, wherein the compacted state of the expansion member is a deflated state of the hole-dilating balloon, wherein the expanding the expansion member comprises inflating the hole-dilating balloon, and wherein the compressing the expansion member comprises deflating the hole-dilating balloon.

Example 100. The method of any example herein, particularly of example 98 or 99, wherein the target tissue is a host leaflet of a host valvular structure.

Example 101. The method of any example herein, particularly of example 100, further comprising, after the retrieving the dilation apparatus, advancing a replacement valve delivery apparatus comprising a guest prosthetic valve, to the host valvular structure.

Example 102. The method of any example herein, particularly of example 101, further comprising positioning the guest prosthetic valve in a radially compressed state thereof within the host valvular structure, and radially expanding the guest prosthetic valve.

Example 103. The method of any example herein, particularly of example 102, wherein the positioning the guest prosthetic valve within the host valvular structure comprises positioning the guest prosthetic valve within the leaflet opening.

Example 104. The method of any example herein, particularly of example 102, wherein the positioning the guest prosthetic valve within the host valvular structure comprises positioning the guest prosthetic valve between host leaflets of the host valvular structure.

Example 105. The method of any example herein, particularly of any one of examples 102 to 104, wherein the radially expanding the guest prosthetic valve comprises inflating a valve-expanding balloon of the replacement valve delivery apparatus, over which the guest prosthetic valve is disposed.

Example 106. The method of any example herein, particularly of any one of examples 102 to 104, wherein the radially expanding the guest prosthetic valve comprises actuating a mechanical actuator of the guest prosthetic valve.

Example 107. The method of any example herein, particularly of any one of examples 102 to 104, wherein the guest prosthetic valve is a self-expandable prosthetic valve, and wherein radially expanding the guest prosthetic valve comprises removing a restraint from around the guest prosthetic valve.

Example 108. The method of any example herein, particularly of any one of examples 52 to 97, wherein the target tissue is a host leaflet of a host valvular structure.

Example 109. The method of any example herein, particularly of any one of examples 100 to 108, wherein the host valvular structure is a valvular structure of a native heart valve.

Example 110. The method of any example herein, particularly of example 109, wherein the native heart valve is an aortic valve.

Example 111. The method of any example herein, particularly of any one of examples 100 to 108, wherein the host valvular structure is a valvular structure of a previously implanted prosthetic valve.

Example 112. A method comprising: advancing a tissue perforation apparatus to a target tissue over a guidewire, wherein the tissue perforation apparatus comprises an anchor device and a needle, and wherein the guidewire extends through an anchor shaft and a helical anchor distal to the anchor shaft of the anchor device; advancing the helical anchor towards a target tissue to anchor the anchor device to the target tissue; distally advancing the guidewire through an anchor channel of the helical anchor; determining that the guidewire does not bend upon contact with the target tissue; and upon determining that the guidewire does not bend, advancing the needle to form a pilot opening at the target tissue.

Example 113. The method of any example herein, particularly of example 112, wherein the determining that the guidewire does not bend comprises determining that the guidewire is not bent radially away from the helical anchor.

Example 114. The method of any example herein, particularly of example 112 or 113, wherein the determining that the guidewire does not bend comprises determining that the guidewire does not exit the helical anchor.

Example 115. The method of any example herein, particularly of any one of examples 112 to 114, wherein the determining that the guidewire does not bend comprises determining that the guidewire does not bend that the portion of the guidewire distal to the anchor shaft is straight.

Example 116. The method of any example herein, particularly of any one of examples 112 to 115, wherein the advancing the tissue perforation apparatus comprises maintaining the anchor device inside a lumen of a delivery catheter of the tissue perforation apparatus.

Example 117. The method of any example herein, particularly of example 116, wherein the advancing the tissue perforation apparatus comprises maintaining the helical anchor proximal to a delivery catheter distal end of the delivery catheter.

Example 118. The method of any example herein, particularly of example 116 or 117, further comprising, prior to the advancing the helical anchor, bringing the delivery catheter distal end to contact with the target tissue.

Example 119. The method of any example herein, particularly of any one of examples 112 to 118, wherein the needle comprises a needle head terminating at a needle tip.

Example 120. The method of any example herein, particularly of example 119, wherein the advancing the tissue perforation apparatus comprises maintaining the needle tip proximal to an anchor tip of the helical anchor.

Example 121. The method of any example herein, particularly of example 119 or 120, further comprising, after the determining, forming a pilot puncture in the target tissue by distally advancing the needle relative to the anchor device.

Example 122. The method of any example herein, particularly of example 121, wherein the forming the pilot puncture further comprises penetrating through the target tissue with the needle head.

Example 123. The method of any example herein, particularly of example 121 or 122, further comprising, after the forming the pilot puncture, retracting the tissue perforation apparatus from the target tissue.

Example 124. The method of any example herein, particularly of example 123, wherein the retracting the tissue perforation apparatus comprises proximally pulling the needle away from the target tissue.

Example 125. The method of any example herein, particularly of example 123 or 124, wherein the retracting the tissue perforation apparatus releasing the helical anchor from the target tissue.

Example 126. The method of any example herein, particularly of example 125, wherein the advancing the helical anchor to anchor the anchor device comprises rotating the anchor device in a first rotational direction.

Example 127. The method of any example herein, particularly of example 126, wherein the releasing the helical anchor comprises rotating the anchor device in a second rotational direction, opposite to the first rotational direction.

Example 128. The method of any example herein, particularly of any one of examples 123 to 127, further comprising, prior to the advancing the tissue perforation apparatus, advancing an outer catheter of a steerable delivery apparatus to the target tissue.

Example 129. The method of any example herein, particularly of example 128, wherein the advancing the tissue perforation apparatus comprises advancing the tissue perforation apparatus through the outer catheter.

Example 130. The method of any example herein, particularly of example 128 or 129, wherein the retracting the tissue perforation apparatus comprises retrieving the anchor device and the needle, while maintaining the outer catheter in position.

Example 131. The method of any example herein, particularly of example 130, further comprising, after the retrieving the anchor device and the needle, advancing a dilation apparatus comprising an expansion member to the target tissue.

Example 132. The method of any example herein, particularly of example 131, wherein the advancing the dilation apparatus comprises advancing the expansion member through the outer catheter.

Example 133. The method of any example herein, particularly of example 131 or 132, further comprising positioning the expansion member inside the pilot puncture, in a compacted state of the expansion member.

Example 134. The method of any example herein, particularly of example 133, further comprising, prior to the positioning the expansion member inside the pilot puncture, passing a dilator of the dilation apparatus through the pilot puncture, thereby further expanding the pilot puncture.

Example 135. The method of any example herein, particularly of example 134, wherein the dilator comprises a dilator tapering portion terminating at a dilator distal end.

Example 136. The method of any example herein, particularly of any one of examples 133 to 135, further comprising forming a tissue opening inside the target tissue by expanding the expansion member.

Example 137. The method of any example herein, particularly of example 136, further comprising, after the forming the tissue opening, compressing the expansion member.

Example 138. The method of any example herein, particularly of example 137, further comprising, after the compressing the expansion member, retrieving the dilation apparatus.

Example 139. The method of any example herein, particularly of example 138, wherein the expansion member is a hole-dilating balloon mounted on a balloon catheter of the dilation apparatus, wherein the compacted state of the expansion member is a deflated state of the hole-dilating balloon, wherein the expanding the expansion member comprises inflating the hole-dilating balloon, and wherein the compressing the expansion member comprises deflating the hole-dilating balloon.

Example 140. The method of any example herein, particularly of example 138 or 139, wherein the target tissue is a host leaflet of a host valvular structure.

Example 141. The method of any example herein, particularly of example 140, further comprising, after the retrieving the dilation apparatus, advancing a replacement valve delivery apparatus comprising a guest prosthetic valve, to the host valvular structure.

Example 142. The method of any example herein, particularly of example 141, further comprising positioning the guest prosthetic valve in a radially compressed state thereof within the host valvular structure, and radially expanding the guest prosthetic valve.

Example 143. The method of any example herein, particularly of example 142, wherein the positioning the guest prosthetic valve within the host valvular structure comprises positioning the guest prosthetic valve within the leaflet opening.

Example 144. The method of any example herein, particularly of example 142, wherein the positioning the guest prosthetic valve within the host valvular structure comprises positioning the guest prosthetic valve between host leaflets of the host valvular structure.

Example 145. The method of any example herein, particularly of any one of examples 142 to 144, wherein the radially expanding the guest prosthetic valve comprises inflating a valve-expanding balloon of the replacement valve delivery apparatus, over which the guest prosthetic valve is disposed.

Example 146. The method of any example herein, particularly of any one of examples 142 to 144, wherein the radially expanding the guest prosthetic valve comprises actuating a mechanical actuator of the guest prosthetic valve.

Example 147. The method of any example herein, particularly of any one of examples 142 to 144, wherein the guest prosthetic valve is a self-expandable prosthetic valve, and wherein radially expanding the guest prosthetic valve comprises removing a restraint from around the guest prosthetic valve.

Example 148. The method of any example herein, particularly of any one of examples 112 to 137, wherein the target tissue is a host leaflet of a host valvular structure.

Example 149. The method of any example herein, particularly of any one of examples 140 to 148, wherein the host valvular structure is a valvular structure of a native heart valve.

Example 150. The method of any example herein, particularly of examples 149, wherein the native heart valve is an aortic valve.

Example 151. The method of any example herein, particularly of any one of examples 140 to 148, wherein the host valvular structure is a valvular structure of a previously implanted prosthetic valve.

It is appreciated that certain features of the disclosure, which are, for clarity, described in the context of separate examples, may also be provided in combination in a single example. Conversely, various features of the disclosure, which are, for brevity, described in the context of a single example, may also be provided separately or in any suitable sub-combination or as suitable in any other described example of the disclosure. No feature described in the context of an example is to be considered an essential feature of that example, unless explicitly specified as such.

In view of the many possible examples to which the principles of the disclosure may be applied, it should be recognized that the illustrated examples are only preferred examples and should not be taken as limiting the scope. Rather, the scope is defined by the following claims. We therefore claim all that comes within the scope and spirit of these claims.

We claim:

1. A tissue perforation apparatus comprising:
an anchor device comprising:
a helical anchor defining an anchor channel and terminating with an anchor tip;
a plunger extending between a plunger distal end portion and a plunger proximal end portion, wherein the plunger is disposed around at least a portion of the helical anchor, and wherein the plunger and the helical anchor are axially movable with respect to each other;
a radiopaque anchor marker configured to move along with the helical anchor; and
a radiopaque plunger marker configured to move along with the plunger.

2. The apparatus of claim 1, wherein the helical anchor is configured to move distally relative to the plunger when the helical anchor is anchored to a target tissue.

3. The apparatus of claim 1, wherein an axial distance between the radiopaque anchor marker and the radiopaque plunger marker is configured to change when the helical anchor is anchored to a target tissue.

4. The apparatus of claim 1, further comprising a biasing member configured to bias the plunger to a distally-biased position relative to the helical anchor.

5. The apparatus of claim 4, wherein the anchor device further comprises an anchor shaft proximal to the helical anchor and a connector disposed between the helical anchor and the anchor shaft, and wherein the anchor shaft defines an anchor lumen which is in communication with the anchor channel.

6. The apparatus of claim 5, wherein the connector comprises a connector distal end portion affixed to the helical anchor, a connector proximal end portion affixed to the anchor shaft, and a connector intermediate portion extending between the connector distal end portion and the connector proximal end portion.

7. The apparatus of claim 6, wherein a distal end of the connector defines a connector distal outer step at a transition between the connector distal end portion and the helical anchor.

8. The apparatus of claim 7, wherein the plunger defines a plunger distal step distal to the connector distal outer step.

9. The apparatus of claim 8, wherein the biasing member is disposed between the plunger distal step and the connector distal outer step.

10. The apparatus of claim 6, wherein the radiopaque anchor marker is located at the connector distal end portion.

11. The apparatus of claim 1, wherein the radiopaque plunger marker is located at the plunger proximal end portion.

12. The apparatus of claim 1, wherein the radiopaque anchor marker and the radiopaque plunger marker are axially continuous with each other in the distally-biased position of the plunger.

13. A method comprising:
advancing a tissue perforation apparatus to a target tissue over a guidewire, wherein the tissue perforation apparatus comprises an anchor device and a needle, wherein the anchor device comprises a radiopaque anchor marker configured to move along with helical anchor of the anchor device, and a radiopaque plunger marker configured to move along with a plunger of the anchor device;
advancing the helical anchor towards a target tissue to anchor the anchor device to the target tissue;
determining that a distance between the radiopaque anchor marker and the radiopaque plunger marker has changed; and
upon determining that the distance between the radiopaque anchor marker and the radiopaque plunger marker has changed, advancing the needle to form a pilot opening at the target tissue.

14. The method of claim 13, wherein the determining that the distance has changed comprises determining that the distance between the radiopaque anchor marker and the radiopaque plunger marker is greater after the advancing the helical anchor than before the advancing the helical anchor.

15. The method of claim 14, wherein the radiopaque anchor marker and the radiopaque plunger marker form a continuous radiopaque image under fluoroscopy, prior to the advancing the helical anchor.

16. The method of claim 13, further comprising, before the advancing the helical anchor, maintaining the plunger in a distally-biased position relative to the helical anchor.

17. The method of claim 16, wherein the anchor device further comprises a biasing member configured to maintain the plunger in the distally-biased position before the anchoring the helical anchor, and wherein the anchoring the helical anchor comprises compressing the biasing member.

18. The method of claim 13, wherein the anchor device further comprises an anchor shaft proximal to the helical anchor, wherein the anchor shaft defines an anchor lumen which is in communication with an anchor channel of the helical anchor, and wherein the advancing the helical anchor to anchor the anchor device comprises rotating the anchor device in a first rotational direction.

19. A method comprising:
advancing a tissue perforation apparatus to a target tissue over a guidewire, wherein the tissue perforation apparatus comprises an anchor device and a needle, and wherein the guidewire extends through an anchor shaft and a helical anchor distal to the anchor shaft of the anchor device;
advancing the helical anchor towards a target tissue to anchor the anchor device to the target tissue;
distally advancing the guidewire through an anchor channel of the helical anchor;
determining that the guidewire does not bend upon contact with the target tissue; and
upon determining that the guidewire does not bend, advancing the needle to form a pilot opening at the target tissue.

20. The method of claim 19, wherein the determining that the guidewire does not bend comprises determining that the guidewire is not bent radially away from the helical anchor.

21. The method of claim 19, wherein the determining that the guidewire does not bend comprises determining that the guidewire does not exit the helical anchor.
